(19) United States
(12) Patent Application Publication
(10) Pub. No.: US 2025/0263471 A1
(43) Pub. Date: Aug. 21, 2025

(54) METHODS OF TREATING ALLERGY USING ANTI-BET V 1 ANTIBODIES

(71) Applicant: Regeneron Pharmaceuticals, Inc., Tarrytown, NY (US)

(72) Inventors: Amanda ATANASIO, Valhalla, NY (US); Deepti R. DESHPANDE, Short Hills, NJ (US); Jennifer MALONEY, Hastings-on-Hudson, NY (US); Jamie M. ORENGO, Cortlandt Manor, NY (US)

(21) Appl. No.: 19/040,236

(22) Filed: Jan. 29, 2025

Related U.S. Application Data
(60) Provisional application No. 63/626,956, filed on Jan. 30, 2024.
(60) Provisional application No. 63/648,321, filed on May 16, 2024.

(51) Int. Cl. C07K 16/16; A61K 39/00; A61P 37/08

(52) U.S. Cl. CPC C07K 16/16; A61P 37/08; A61K 2039/507; A61K 2039/545

(57) ABSTRACT

The present disclosure provides methods for treating, preventing, or ameliorating one or more symptoms of birch allergy or allergic disease in a subject by administering to the subject two antibodies or antigen-binding fragments thereof that bind Bet v 1, or a cocktail of two antibodies or antigen-binding fragments thereof that bind Bet v 1.

### CROSS-REFERENCE TO RELATED APPLICATIONS

This application claims priority to U.S. Provisional Patent Application Nos. 63/626,956 filed Jan. 30, 2024, and 63/648,321 filed May 16, 2024, the entire contents of each of which are incorporated by reference herein.

### FIELD OF THE INVENTION

The present disclosure relates to the use of human antibodies that bind to Bet v 1 to treat or prevent allergic reactions and allergic diseases in a subject in need thereof.

### SEQUENCE LISTING

A copy of the sequence listing is submitted concurrently with the specification electronically via Patent Center. The content of the electronic sequence listing (11714US01_Sequence_Listing_ST.26.xml; Size 40,960 bytes; and Date of Creation: Jan. 28, 2025) is herein incorporated by reference in its entirety.

### BACKGROUND

Birch pollen-induced allergic rhinitis with or without conjunctivitis is common in Europe and North America, with clinically relevant sensitization to birch pollen affecting approximately 8% to 16% of the overall population (Chan-Yeung et al., Allergy 2010, 65:1404-1413; Salo et al., J Allergy Clin Immunol 2014, 134:350-359; and Biedermann et al., J. Allergy Clin Immunol 2019, 143:1058-1066) and approximately 20% to 30% of the population with allergic rhinitis (see, e.g., Pablos et al, Current Allergy and Asthma Reports 2016, 16:31). Regardless of the specific allergen, symptoms in allergic rhinoconjunctivitis include rhinorrhea, nasal congestion, nasal itching, sneezing, as well as ocular itching/redness/gritty feeling and eye tearing/watering. These symptoms can result in time away from work/school, medication usage, and overall reduced quality of life (Dykewicz et al., J Allergy Clin Immunol 2020, 146:721-767), and similar is expected for patients with birch allergy. Birch pollen contains a mix of allergenic and non-allergenic proteins; Bet v 1 is the most abundant allergenic pollen protein (Erler et al, Proteomics 2011 11:1486-1498; Schenk et al, Journal of Proteomics 2011, 74:1290-1300). Sensitization rates to Bet v 1 among birch-allergic individuals reach >95%.

For birch-allergic patients, allergen avoidance is challenging in birch endemic areas. Antihistamines and intranasal corticosteroids (INCS) are the standard of care for the treatment of allergic rhinoconjunctivitis regardless of the specific allergen inducing the symptoms, including for patients with birch pollen allergic disease. While the antihistamines are only modestly effective, INCS are more effective at treating nasal symptoms; however, they are less effective for allergic eye symptoms (see, e.g., Ciprandi et al., Current Medical Research and Opinion 2011, 27:1005-1011; and Dykewicz, supra). Most ocular pharmacotherapeutics have limited efficacy for both ocular symptoms of redness and itching, have shorter duration of action, and have the need for frequent use and issues with adherence (Bielory et al., Annals of allergy, asthma & immunology 2020, 124:118-134; Cheung et al., Ophthalmology 2024, 131:P134-:204). Patients with uncontrolled symptoms on standard of care pharmacotherapy, including those with birch pollen-induced allergic rhinitis, are recommended allergen specific immunotherapy (SIT) (Dykewicz, supra). Although disease-modifying, SIT can have several practical limitations including safety with a risk of local and systemic allergic reactions (including potentially life-threatening anaphylaxis), variable response and tolerability, challenges with time commitment and potential issues with poor patient adherence (see, e.g., Roberts et al., Allergy 2018, 73:765-798). Accordingly, there remains a need for safe and effective therapies for treating people with birch allergy.

### BRIEF SUMMARY

In one aspect, the disclosure provides methods of treating birch allergy, or methods of treating one or more symptoms of birch allergy such as one or more symptoms of allergic conjunctivitis or one or more symptoms of allergic rhinitis, in a subject in need thereof. In some embodiments, the method comprises administering to the subject:
(a) a first anti-Bet v 1 antibody or antigen-binding fragment thereof, wherein the first anti-Bet v 1 antibody or antigen-binding fragment thereof comprises a heavy chain complementarity determining region (HCDR) 1 comprising the amino acid sequence of SEQ ID NO:2, an HCDR2 comprising the amino acid sequence of SEQ ID NO:3, an HCDR3 comprising the amino acid sequence of SEQ ID NO:4, a light chain complementarity determining region (LCDR) 1 comprising the amino acid sequence of SEQ ID NO:6, an LCDR2 comprising the amino acid sequence of SEQ ID NO:7, and an LCDR3 comprising the amino acid sequence of SEQ ID NO: 8 (e.g., REGN5713); and
(b) a second anti-Bet v 1 antibody or antigen-binding fragment thereof, wherein the second anti-Bet v 1 antibody or antigen-binding fragment thereof comprises an HCDR1 comprising the amino acid sequence of SEQ ID NO:22, an HCDR2 comprising the amino acid sequence of SEQ ID NO:23, an HCDR3 comprising the amino acid sequence of SEQ ID NO:24, an LCDR1 comprising the amino acid sequence of SEQ ID NO:26, an LCDR2 comprising the amino acid sequence of SEQ ID NO:27, and an LCDR3 comprising the amino acid sequence of SEQ ID NO: 28 (e.g., REGN5715).

In some embodiments, the method does not comprise administering a third anti-Bet v 1 antibody to the subject. In some embodiments, the method does not comprise administering REGN5714 to the subject.

In another aspect, methods of reducing one or more symptoms of an allergic reaction to a Fagales allergen in a subject are provided. In some embodiments, the method comprises administering to the subject:
(a) a first anti-Bet v 1 antibody or antigen-binding fragment thereof, wherein the first anti-Bet v 1 antibody or antigen-binding fragment thereof comprises an HCDR1 comprising the amino acid sequence of SEQ ID NO:2, an HCDR2 comprising the amino acid sequence of SEQ ID NO: 3, an HCDR3 comprising the amino acid sequence of SEQ ID NO:4, an LCDR1 comprising the amino acid sequence of SEQ ID NO:6, an LCDR2 comprising the amino acid sequence of SEQ ID NO:7, and an LCDR3 comprising the amino acid sequence of SEQ ID NO:8 (e.g., REGN5713); and
(b) a second anti-Bet v 1 antibody or antigen-binding fragment thereof, wherein the second anti-Bet v 1 antibody or antigen-binding fragment thereof comprises an HCDR1 comprising the amino acid sequence of SEQ ID NO:22, an HCDR2 comprising the amino acid sequence of SEQ ID NO:23, an HCDR3 comprising the amino acid sequence of SEQ ID NO:24, an LCDR1 comprising the amino acid sequence of SEQ ID NO:26, an LCDR2 comprising the amino acid sequence of SEQ ID NO:27, and an LCDR3 comprising the amino acid sequence of SEQ ID NO: 28 (e.g., REGN5715).

In some embodiments, the method does not comprise administering a third anti-Bet v 1 antibody to the subject. In some embodiments, the method does not comprise administering REGN5714 to the subject.

In some embodiments, the Fagales allergen is Bet v 1. In some embodiments, the subject is sensitized to Bet v 1 and to at least one other Fagales allergen. In some embodiments, the at least one other Fagales allergen is alder, hazel, oak, hornbeam, hop-hornbeam, beech, chestnut, hazelnut, or apple.

In another aspect, methods of treating a subject having seasonal or perennial allergy associated with birch and cross-reacting pollens are provided. In some embodiments, the subject has moderate-to-severe seasonal allergy or moderate-to-severe perennial allergy. In some embodiments, the method comprises administering to the subject:
(a) a first anti-Bet v 1 antibody or antigen-binding fragment thereof, wherein the first anti-Bet v 1 antibody or antigen-binding fragment thereof comprises an HCDR1 comprising the amino acid sequence of SEQ ID NO:2, an HCDR2 comprising the amino acid sequence of SEQ ID NO: 3, an HCDR3 comprising the amino acid sequence of SEQ ID NO:4, an LCDR1 comprising the amino acid sequence of SEQ ID NO:6, an LCDR2 comprising the amino acid sequence of SEQ ID NO:7, and an LCDR3 comprising the amino acid sequence of SEQ ID NO:8 (e.g., REGN5713); and
(b) a second anti-Bet v 1 antibody or antigen-binding fragment thereof, wherein the second anti-Bet v 1 antibody or antigen-binding fragment thereof comprises an HCDR1 comprising the amino acid sequence of SEQ ID NO:22, an HCDR2 comprising the amino acid sequence of SEQ ID NO:23, an HCDR3 comprising the amino acid sequence of SEQ ID NO:24, an LCDR1 comprising the amino acid sequence of SEQ ID NO:26, an LCDR2 comprising the amino acid sequence of SEQ ID NO:27, and an LCDR3 comprising the amino acid sequence of SEQ ID NO: 28 (e.g., REGN5715).

In some embodiments, the method does not comprise administering a third anti-Bet v 1 antibody to the subject. In some embodiments, the method comprises administering no more than two anti-Bet v 1 antibodies to the subject. In some embodiments, the method does not comprise administering REGN5714 to the subject.

In some embodiments, the method comprises administering to the subject a single dose of each of the first anti-Bet v 1 antibody or antigen-binding fragment thereof and the second anti-Bet v 1 antibody or antigen-binding fragment thereof prior to the start of birch pollen season. In some embodiments, the single dose is administered at least 1 week, 2 weeks, 3 weeks, or 4 weeks prior to the start of birch pollen season.

In some embodiments, each of the first anti-Bet v 1 antibody or antigen-binding fragment thereof and the second anti-Bet v 1 antibody or antigen-binding fragment thereof is administered at a dose of about 150 mg to about 300 mg. In some embodiments, each of the first anti-Bet v 1 antibody or antigen-binding fragment thereof and the second anti-Bet v 1 antibody or antigen-binding fragment thereof is administered at a dose of about 150 mg to about 350 mg, e.g., about 150 mg, about 175 mg, about 200 mg, about 225 mg, about 250 mg, about 275 mg, about 300 mg, about 325 mg, or about 350 mg.

In some embodiments, the first anti-Bet v 1 antibody or antigen-binding fragment thereof and the second anti-Bet v 1 antibody or antigen-binding fragment thereof are formulated separately. In some embodiments, the first anti-Bet v 1 antibody or antigen-binding fragment thereof and the second anti-Bet v 1 antibody or antigen-binding fragment thereof are co-formulated in a pharmaceutical composition. In some embodiments, the pharmaceutical composition consists essentially of the first anti-Bet v 1 antibody and the second anti-Bet v 1 antibody.

For the methods disclosed herein, in some embodiments the pharmaceutical composition comprises the first anti-Bet v 1 antibody or antigen-binding fragment thereof and the second anti-Bet v 1 antibody or antigen-binding fragment thereof. In some embodiments the pharmaceutical composition consists essentially of the first anti-Bet v 1 antibody and the second anti-Bet v 1 antibody. In some embodiments the pharmaceutical composition consists of the first anti-Bet v 1 antibody and the second anti-Bet v 1 antibody as active therapeutic molecules, in addition to pharmaceutically acceptable ingredients and/or diluents. In some embodiments the pharmaceutical composition comprises the first anti-Bet v 1 antibody and the second anti-Bet v 1 antibody, and excludes a third anti-Bet v 1 antibody, for example, the REGN5714 antibody, an anti-Bet v 1 antibody comprising an HCDR1 comprising the amino acid sequence of SEQ ID NO: 12, an HCDR2 comprising the amino acid sequence of SEQ ID NO:13, an HCDR3 comprising the amino acid sequence of SEQ ID NO:14, an LCDR1 comprising the amino acid sequence of SEQ ID NO:16, an LCDR2 comprising the amino acid sequence of SEQ ID NO:17, and an LCDR3 comprising the amino acid sequence of SEQ ID NO:18.

In some embodiments, the anti-Bet v 1 antibodies or antigen-binding fragments thereof are provided in a single pharmaceutical composition. In some embodiments, the anti-Bet v 1 antibodies or antigen-binding fragments thereof are provided in more than one pharmaceutical composition, e.g., each anti-Bet v 1 antibody in a separate pharmaceutical composition.

In some embodiments, the pharmaceutical composition(s) comprises the anti-Bet v 1 antibodies (e.g., each of the first anti-Bet v 1 antibody and the second anti-Bet v 1 antibody) or antigen-binding fragments thereof at a total dose (i.e., the total dose of both antibodies) of from about 300 mg to about 600 mg. In some embodiments, the pharmaceutical composition(s) comprises the anti-Bet v 1 antibodies (e.g., each of the first anti-Bet v 1 antibody and the second anti-Bet v 1 antibody) at a total dose of about 300 mg, e.g., about 150 mg of the first antibody and about 150 mg of the second antibody. In some embodiments, the pharmaceutical composition(s) comprises the anti-Bet v 1 antibodies (e.g., each of the first anti-Bet v 1 antibody and the second anti-Bet v 1 antibody) at a total dose of about 600 e.g., about 300 mg of the first antibody and about 300 mg of the second antibody mg.

In some embodiments, the anti-Bet v 1 antibodies or antigen-binding fragments thereof, or the pharmaceutical composition comprising the anti-Bet v 1 antibodies, are administered subcutaneously. In some embodiments, the anti-Bet v 1 antibodies, or the pharmaceutical composition comprising the anti-Bet v 1 antibodies, are administered intravenously.

In some embodiments, a single dose of the anti-Bet v 1 antibodies or antigen-binding fragments thereof, or the pharmaceutical composition comprising the anti-Bet v 1 antibodies or antigen-binding fragments thereof, are administered. In some embodiments, the anti-Bet v 1 antibodies, or the pharmaceutical composition comprising the anti-Bet v 1 antibodies, are administered once before the start of pollen season.

In some embodiments, the first anti-Bet v 1 antibody or antigen-binding fragment thereof comprises a heavy chain variable region (HCVR) comprising the amino acid sequence of SEQ ID NO:1 and a light chain variable region (LCVR) comprising the amino acid sequence of SEQ ID NO:5. In some embodiments, the first anti-Bet v 1 antibody comprises a heavy chain comprising the amino acid sequence of SEQ ID NO:9 and a light chain comprising the amino acid sequence of SEQ ID NO:10.

In some embodiments, the second anti-Bet v 1 antibody or antigen-binding fragment thereof comprises an HCVR comprising the amino acid sequence of SEQ ID NO:21 and an LCVR comprising the amino acid sequence of SEQ ID NO:25. In some embodiments, the second anti-Bet v 1 antibody comprises a heavy chain comprising the amino acid sequence of SEQ ID NO:29 and a light chain comprising the amino acid sequence of SEQ ID NO:30.

In some embodiments, the pharmaceutical composition excludes an anti-Bet v 1 antibody comprising an HCVR comprising the amino acid sequence of SEQ ID NO:11 and an LCVR comprising the amino acid sequence of SEQ ID NO:15. In some embodiments, the pharmaceutical composition excludes an anti-Bet v 1 antibody comprising a heavy chain comprising the amino acid sequence of SEQ ID NO:19 and a light chain comprising the amino acid sequence of SEQ ID NO:20.

In some embodiments, treatment with the pharmaceutical composition:
reduces a subject's Total Nasal Symptom Score (TNSS);
reduces a subject's Total Ocular Symptom Score (TOSS);
reduces a subject's Total Symptom Score (TSS);
reduces a subject's Daily Medication Score (DMS);
reduces a subject's Combined Symptom and Medication Score (CSMS);
reduces a subject's birch skin prick test (SPT) mean wheal diameter; and/or
increases a subject's number of “well days” in which rescue medication is not utilized and the subject's TSS is ≤2 of 18.

In some embodiments, the TNSS, TOSS, TSS, DMS, CSMS, SPT mean wheal diameter, and/or number of well days is measured over at least 28, 57, or 85 days. In some embodiments, the TNSS, TOSS, TSS, DMS, CSMS, SPT mean wheal diameter, and/or number of well days is measured over the duration of birch pollen season.

In some embodiments, treatment with the pharmaceutical composition reduces allergic rhinitis symptoms in the subject. In some embodiments, treatment with the pharmaceutical composition reduces a subject's mean Total Nasal Symptom Score (TNSS) by at least about 39.5% from placebo 29 days or 1 month after receiving the pharmaceutical composition. In some embodiments, treatment with the pharmaceutical composition reduces a subject's mean TNSS versus placebo by about-2.4 at about day 57 or around 2 months after receiving treatment; in some embodiments, treatment with the pharmaceutical composition reduces a subject's mean TNSS versus placebo by about-1.66 at about day 85 or around 3 months after receiving treatment.

In some embodiments, administration of a single dose of the pharmaceutical composition reduces a subject's mean TNSS by at least about 20% for at least two months after the pharmaceutical composition is administered; and/or reduces a subject's mean TNSS by at least about 25% for at least two months after the pharmaceutical composition is administered.

In some embodiments, treatment with the pharmaceutical composition reduces allergic conjunctivitis symptoms in the subject. In some embodiments, treatment with the pharmaceutical composition: reduces a subject's Total Ocular Symptom Score (TOSS) (e.g., by at least about 15%, 20%, 25%, 30%, 35%, 40%, 45%, 50% or more), relative to a baseline TOSS value for the subject prior to the onset of treatment; and/or reduces a subject's TOSS AUC (0-1 hr) after NAC (e.g., by at least about 15%, 20%, 25%, 30%, 35%, 40%, 45%, 50% or more), relative to a baseline TOSS AUC (0-1 hr) value after NAC for the subject prior to the onset of treatment.

In some embodiments, treatment with the pharmaceutical composition reduces a subject's combined symptom and medication score (CSMS) during birch pollen season (e.g., by at least about 10%, 15%, 20%, 25%, 30%, 35%, 40%, 45%, 50% or more), relative to a baseline CSMS for the subject prior to the onset of treatment or a control CSMS.

In some embodiments, treatment with the pharmaceutical composition improves peak nasal inspiratory flow (PNIF) in the subject (e.g., by at least about 25%, 30%, 35%, 40%, 45%, 50%, 55%, 60%, 65%, 70%, 75% or more), relative to a baseline PNIF value for the subject prior to the onset of treatment.

In some embodiments, treatment with the pharmaceutical composition reduces birch sensitization in the subject (e.g., by at least about 30%, 40%, 50%, 60%, 70%, 80%, 90% or more) as measured by a skin prick test (SPT) with a birch allergen extract. In some embodiments, administration of a single dose of the pharmaceutical composition reduces birch sensitization in the subject by at least about 60% or more for at least two months (e.g., at least three months, at least four months, at least five months, or at least six months) after the pharmaceutical composition is administered.

In some embodiments, the subject to be treated has a baseline serum allergen-specific IgE level ≥0.35 kUa/L for the allergen (e.g., birch tree pollen, Bet v 1 allergen, or Fagales allergen). In some embodiments, the subject to be treated has a baseline serum birch and Bet v 1 sIgE level ≥0.7 kUa/L. In some embodiments, the subject to be treated has a baseline positive SPT with an allergen (e.g., birch allergen extract or Fagales allergen), for example, a birch SPT of greater than or equal to 5 mm. In some embodiments, the subject to be treated has at least a 6 (in a range of 0 to 12) TNSS at ≥2 timepoints in screening.

In some embodiments, the subject to be treated has a baseline ocular itch score ≥2 in both eyes after a conjunctival allergen challenge (CAC), as measured using the Ora Calibra® Conjunctival Allergen Challenge Ocular Itching Scale. In some embodiments, the subject to be treated has a baseline conjunctival redness score ≥2 in both eyes after a CAC, as measured using the Ora Calibra® Ocular Hyperemia Scale.

In another aspect, the disclosure provides a first anti-Bet v 1 antibody or antigen-binding fragment thereof (e.g., REGN5713) and a second anti-Bet v 1 antibody or antigen-binding fragment thereof (e.g., REGN5715), or a cocktail comprising the two anti-Bet v 1 antibodies or antigen-binding fragments thereof for use in a method of treating birch allergy in a subject. In some embodiments, the method comprises administering the two anti-Bet v 1 antibodies or antigen-binding fragments thereof or the cocktail as disclosed herein to a subject in need thereof (e.g., a subject having birch allergy).

In another aspect, the use of a first anti-Bet v 1 antibody or antigen-binding fragment thereof (e.g., REGN5713) and a second anti-Bet v 1 antibody or antigen-binding fragment thereof (e.g., REGN5715), or a cocktail comprising the two anti-Bet v 1 antibodies or antigen-binding fragments thereof, in the manufacture of a medicament for use in a method of treating birch allergy in a subject is provided. In some embodiments, the method comprises administering the medicament as disclosed herein to a subject in need thereof (e.g., a subject having birch allergy).

In another aspect, the disclosure provides a first anti-Bet v 1 antibody or antigen-binding fragment thereof (e.g., REGN5713) and a second anti-Bet v 1 antibody or antigen-binding fragment thereof (e.g., REGN5715), or a cocktail comprising the two anti-Bet v 1 antibodies or antigen-binding fragments thereof for use in a method of reducing one or more symptoms of an allergic reaction to a Fagales allergen in a subject. In some embodiments, the method comprises administering the two anti-Bet v 1 antibodies or antigen-binding fragments thereof or the cocktail as disclosed herein to a subject in need thereof (e.g., a subject having one or more symptoms of an allergic reaction to a Fagales allergen).

In another aspect, the use of a first anti-Bet v 1 antibody or antigen-binding fragment thereof (e.g., REGN5713) and a second anti-Bet v 1 antibody or antigen-binding fragment thereof (e.g., REGN5715), or a cocktail comprising the two anti-Bet v 1 antibodies or antigen-binding fragments thereof, in the manufacture of a medicament for use in a method of reducing one or more symptoms of an allergic reaction to a Fagales allergen in a subject is provided. In some embodiments, the method comprises administering the medicament as disclosed herein to a subject in need thereof (e.g., a subject having one or more symptoms of an allergic reaction to a Fagales allergen).

In another aspect, the disclosure provides a first anti-Bet v 1 antibody or antigen-binding fragment thereof (e.g., REGN5713) and a second anti-Bet v 1 antibody or antigen-binding fragment thereof (e.g., REGN5715), or a cocktail comprising the two anti-Bet v 1 antibodies or antigen-binding fragments thereof for use in a method of treating a subject having seasonal or perennial allergy associated with birch and cross-reacting pollens. In some embodiments, the method comprises administering the two anti-Bet v 1 antibodies or antigen-binding fragments thereof or the cocktail as disclosed herein to a subject in need thereof (e.g., a subject having seasonal or perennial allergy associated with birch and cross-reacting pollens are provided).

In another aspect, the use of a first anti-Bet v 1 antibody or antigen-binding fragment thereof (e.g., REGN5713) and a second anti-Bet v 1 antibody or antigen-binding fragment thereof (e.g., REGN5715), or a cocktail comprising the two anti-Bet v 1 antibodies or antigen-binding fragments thereof, in the manufacture of a medicament for use in a method of treating a subject having seasonal or perennial allergy associated with birch and cross-reacting pollens are provided. In some embodiments, the method comprises administering the medicament as disclosed herein to a subject in need thereof (e.g., a subject having seasonal or perennial allergy associated with birch and cross-reacting pollens are provided).

Other embodiments will be apparent from a review of the ensuing detailed description.

### DETAILED DESCRIPTION

### Definitions

Before the present invention is described, it is to be understood that the invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

The term “Bet v 1”, as used herein, refers to a Bet v 1 protein, either in natural/native form or recombinantly produced. The natural Bet v 1 protein is approximately 17 kD and exists as a 7 stranded anti-parallel β-sheet (β1-β7), two short α-helices (α1 and α2) connecting β1 and β2, a long C-terminal α-helix (α3), and the glycine-rich loop motif between β2 and β3 (Kofler et al., J. Mol. Biol. 2012, 422(1): 109-123). In some embodiments, a Bet v 1 protein comprises the amino acid sequence of SEQ ID NO:31. In some embodiments, a Bet v 1 protein comprises a naturally occurring or recombinantly produced form that comprises one or more amino acid substitutions, deletions, or additions relative to SEQ ID NO:31. For example, in some embodiments, a Bet v 1 protein comprises the amino acid sequence of SEQ ID NO:32 (the Bet v 1 amino acid sequence from Uniprot: P15494).

The term “Bet v 1 fragment,” as used herein, refers to a polypeptide having at least one antigenic site of Bet v 1. In some embodiments, a Bet v 1 fragment is a polypeptide having at least two antigen sites of Bet v 1. In some embodiments, the antigenic sites are covalently linked. In some embodiments, the antigenic sites are linked by at least one peptide bond. In one embodiment, the two antigenic sites are linked by at least one peptide bond and a spacer between the antigenic sites. Exemplary Bet v 1 fragments are disclosed in WO 2018/222854, incorporated by reference herein.

The term “antibody,” as used herein, refers to an antigen-binding molecule or molecular complex comprising a set of complementarity determining regions (CDRs) that specifically bind to or interact with a particular antigen (e.g., Bet v 1). The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, the present disclosure includes methods comprising the use of bispecific antibodies wherein one arm of an immunoglobulin is specific for Bet v 1 or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Specific binding can be characterized by an equilibrium dissociation constant of at least about 1×10⁻⁶ M or less (e.g., a smaller K_{D} denotes a tighter binding). Methods for determining whether an antibody specifically binds to an antigen are well known in the art and include, for example, equilibrium dialysis, surface plasmon resonance, and the like. In some embodiments, specific binding is measured in a surface plasmon resonance assay. An isolated antibody that specifically binds an antigen from one species may or may not have cross-reactivity to other antigens, such as an orthologous antigen from another species.

The term “K_{D},” as used herein, refers to the equilibrium dissociation constant of a particular antibody-antigen interaction.

The term “surface plasmon resonance,” as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The term “epitope,” as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be either linear or discontinuous (e.g., conformational). A linear epitope is one produced by adjacent amino acid residues in a polypeptide chain. A conformational epitope is produced by spatially juxtaposed amino acids from different segments of the linear polypeptide chain. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes may also be defined as structural or functional. Functional epitopes are generally a subset of the structural epitopes and have those residues that directly contribute to the affinity of the interaction. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or at least 8-10 amino acids in a unique spatial conformation.

The terms “substantial identity” and “substantially identical,” as used with reference to a nucleic acid or fragment thereof, indicates that, when optimally aligned with appropriate nucleotide insertions or deletions with another nucleic acid (or its complementary strand), there is nucleotide sequence identity in at least about 90%, e.g., at least 91%, 92%, 93%, 94%, 95%, 96%, 97%, 98% or 99%, of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the terms “substantial identity” and “substantially identical” mean that two peptide sequences, when optimally aligned, share at least about 90% sequence identity, e.g., at least 90%, 91%, 92%, 93%, 94%, 95%, 96%, 97%, 98% or 99% sequence identity. In some embodiments, residue positions that are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild-type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson, 2000 supra). Another preferred algorithm when comparing a sequence of the present disclosure to a database containing a large number of sequences from different organisms is the computer program BLAST, especially BLASTP or TBLASTN, using default parameters. (See, e.g., Altschul et al., 1990, J. Mol. Biol. 215:403-410 and 1997 Nucleic Acids Res. 25:3389-3402).

As used herein, the terms “allergic response,” “allergic reaction,” “allergic symptom,” and the like, include one or more signs or symptoms selected from the group consisting of urticaria (e.g., hives), angioedema, rhinitis, asthma, vomiting, sneezing, runny nose, sinus inflammation, watery eyes, wheezing, bronchospasm, reduced peak expiratory flow (PEF), gastrointestinal distress, flushing, swollen lips, swollen tongue, reduced blood pressure, anaphylaxis, and organ dysfunction/failure. An “allergic response,” “allergic reaction,” “allergic symptom,” etc., also includes immunological responses and reactions such as, e.g., increased IgE production and/or increased allergen-specific immunoglobulin production.

The term “allergen” refers to a substance, chemical, particle or composition that is capable of stimulating an allergic response in a susceptible individual. Allergens may be contained within or derived from a food item such as, e.g., dairy products (e.g., cow's milk), egg, celery, sesame, wheat, soy, fish, shellfish, sugars (e.g., sugars present on meat such as alpha-galactose), peanuts, other legumes (e.g., beans, peas, soybeans, etc.), and tree nuts. Alternatively, an allergen may be contained within or derived from a non-food item such as, e.g., dust (e.g., containing dust mite), pollen, insect venom (e.g., venom of bees, wasps, mosquitos, fire ants, etc.), mold, animal fur, animal dander, wool, latex, metals (e.g., nickel), household cleaners, detergents, medication, cosmetics (e.g., perfumes, etc.), drugs (e.g., penicillin, sulfonamides, salicylate, etc.), therapeutic monoclonal antibodies (e.g., cetuximab), ragweed, grass and birch. In some embodiments, an allergen is birch pollen or is contained within or derived from birch, e.g., a Bet v 1 protein. The terms “allergen” and “antigen” are used interchangeably through the disclosure.

In some embodiments, “birch pollen season” is defined as a time period starting with the first of 3 consecutive days when the birch pollen count is 10 grains/m³ or greater in a given geographic area and ending on the last day of the last occurrence of 3 consecutive days with a pollen count of 10 grains/m³ or greater. In some embodiments, “peak birch pollen season” is defined as the 15 consecutive days within the birch pollen season having the highest 15-day moving average pollen count.

As used herein, the term “subject in need thereof” refers to a human or non-human mammal that (i) exhibits one or more symptoms or indicia of allergy (e.g., birch allergy), (ii) has been diagnosed with allergy to an allergen (e.g., birch pollen allergen); and/or (iii) is at an increased risk for developing an allergy or an allergic response to an allergen (e.g., birch allergy or allergic response). In certain embodiments, the term includes subjects that show allergen sensitization to one or more allergens (e.g., birch allergens or a component thereof such as Bet v 1 protein). In some embodiments, a subject is sensitized to an allergen (e.g., birch allergen or Bet v 1 protein) if the subject exhibits a level of allergen-specific IgE for the allergen that is ≥ 0.35 kUa/L. In some embodiments, a subject in need thereof is a subject who has a level of allergen-specific IgE for birch allergen that is ≥0.7 kUa/L. In certain embodiments, a subject in need of treatment according to the methods of the present disclosure is a subject having an elevated level of one or more serum biomarkers including, but not limited to, total IgE, allergen-specific IgE (e.g., birch pollen IgE or Bet v 1 IgE), thymus and activation-regulated chemokine (TARC), and eotaxin. For example, in some embodiments, the methods of the present disclosure comprise administering the anti-Bet v 1 antibodies individually or the REGN5713/REGN5715 antibody cocktail to patients with elevated levels of allergen-specific IgE (e.g., a subject having a birch pollen or Bet v 1 IgE level ≥0.35 kUa/L or ≥0.7 kUa/L). The terms “subject” and “patient” are used interchangeably herein.

The term “subject in need thereof” may also include, e.g., subjects who have a concomitant allergy or other condition. For example, in some embodiments a subject having a birch allergy may also have oral allergy syndrome. In some embodiments, a subject to be treated is a subject having a birch allergy and an allergy to one or more other Fagales order allergens. Fagales order allergens, or “Fagales allergens,” as used herein, include but are not limited to birch pollen (Bet v 1), alder pollen (Aln g1 and Aln g4), hazel pollen (Cor a1, Cor a2, Cor a8, Cor a9, Cor a10, Cor a11, Cor a12, Cor a13, and Cor a14), hornbeam pollen (Car b1), hop-hornbeam pollen (Ost c1), chestnut pollen (Cas s1, Cas s5, Cas s8, and Cas s9), beech pollen (Fag s1) and white oak pollen (Que a1 and Que a2). A person of skill in the art will recognize that Bet v 1-related allergens (also referred to as “Fagales group 1” allergens or “PR-10 allergens”) are also found in foods such as apple (Mal d 1), apricot (Pru ar 1), carrot (Dau c 1), celery (Api g 1), cherry (Pru av 1), chestnut (Cas s 1), hazelnut (Cor a 1), kiwi (Act c 8, Act d 8, and Act d 11), mungbean (Vig r 1), peanut (Ara h 8), pear (Pyr c 1), raspberry (Rub i 1), soybean (Gly m 4), strawberry (Fra a 1), tomato (Sola l 4), and walnut (Jug r 5). See, Carlson, Annals of Allergy, Asthma & Immunology 2019, 123:P359-365. Thus, the term “Fagales allergen” includes not only pollen allergens but also food allergens. In some embodiments, the subject has an elevated level of allergen-specific IgE (e.g., an allergen-specific IgE level ≥0.35 kUa/L or ≥0.7 kUa/L) to birch pollen (e.g., birch pollen extract) or a Bet v 1 allergen and to one or more other Fagales allergens. In some embodiments, the subject has an elevated level of allergen-specific IgE (e.g., an allergen-specific IgE level ≥0.35 kUa/L or ≥0.7 kUa/L) to birch pollen (e.g., birch pollen extract) or a Bet v 1 allergen and to oak pollen (e.g., Que a1 and/or Que a2).

### INTRODUCTION

As described herein, monoclonal antibodies have been developed against Bet v 1, the major birch tree allergen. It has been hypothesized that high-affinity allergen-specific monoclonal IgG antibodies can be administered as a form of providing “passive immunity” to the allergen. It has been previously shown that the anti-Bet v 1 antibodies REGN5713, REGN5714, and REGN5715 bind independently and non-competitively to the Bet v 1 allergen, and effectively block Bet v 1 binding to birch-allergic polyclonal IgE and prevent the Bet v 1- and BPE-induced allergic response (Atanasio et al, J Allergy Clin Immunol 2022, 149:200-211).

As described in the Examples section below, it has been surprisingly discovered that a combination of two anti-Bet v 1 antibodies, REGN5713 and REGN5715, is as efficacious in reducing symptoms of birch allergy as a three-antibody combination of REGN5713, 5714, and 5715. Moreover, administration of a single dose of each of REGN5713 and REGN5715 provides as durable a response as the three-antibody combination. The ability to use two antibodies instead of three antibodies for the treatment of birch allergic patients offers advantages such as greater ease of administration due to the ability to use a lower volume for administration, potential for fewer adverse effects or reactions to the antibodies, and greater ease of co-formulating the antibodies.

### Therapeutic Methods

In one aspect, methods for treating birch allergy or for treating, preventing, or ameliorating one or more symptoms of birch allergy in a subject are provided. In another aspect, methods for treating, preventing, or ameliorating seasonal or perennial allergy (e.g., moderate to severe seasonal or perennial allergy) to birch and/or birch cross-reacting pollens are provided.

In some embodiments, the methods comprise administering to the subject a single dose of each of the first anti-Bet v 1 antibody or antigen-binding fragment thereof and the second anti-Bet v 1 antibody or antigen-binding fragment thereof, or a pharmaceutical composition comprising the first and second anti-Bet v 1 antibodies or antigen-binding fragments thereof, prior to the start of birch pollen season. In some embodiments, the single dose is administered at least 1 week, 2 weeks, 3 weeks, or 4 weeks prior to the start of birch pollen season.

In some embodiments, the methods comprise administering to the subject a first anti-Bet v 1 antibody or antigen-binding fragment thereof, wherein the first anti-Bet v 1 antibody comprises a heavy chain complementarity determining region (HCDR) 1 comprising the amino acid sequence of SEQ ID NO:2, an HCDR2 comprising the amino acid sequence of SEQ ID NO: 3, an HCDR3 comprising the amino acid sequence of SEQ ID NO:4, a light chain complementarity determining region (LCDR) 1 comprising the amino acid sequence of SEQ ID NO: 6, an LCDR2 comprising the amino acid sequence of SEQ ID NO:7, and an LCDR3 comprising the amino acid sequence of SEQ ID NO:8 (e.g., REGN5713); and a second anti-Bet v 1 antibody or antigen-binding fragment thereof, wherein the second anti-Bet v 1 antibody comprises an HCDR1 comprising the amino acid sequence of SEQ ID NO:22, an HCDR2 comprising the amino acid sequence of SEQ ID NO:23, an HCDR3 comprising the amino acid sequence of SEQ ID NO:24, an LCDR1 comprising the amino acid sequence of SEQ ID NO:26, an LCDR2 comprising the amino acid sequence of SEQ ID NO:27, and an LCDR3 comprising the amino acid sequence of SEQ ID NO:28 (e.g., REGN5715); wherein the method does not comprise administering a third anti-Bet v 1 antibody to the subject. In some embodiments, the method does not comprise administering an anti-Bet v 1 antibody comprising an HCDR1 comprising the amino acid sequence of SEQ ID NO:12, an HCDR2 comprising the amino acid sequence of SEQ ID NO:13, an HCDR3 comprising the amino acid sequence of SEQ ID NO:14, an LCDR1 comprising the amino acid sequence of SEQ ID NO:16, an LCDR2 comprising the amino acid sequence of SEQ ID NO:17, and an LCDR3 comprising the amino acid sequence of SEQ ID NO:18 (e.g., REGN5714).

In some embodiments, the method comprises administering a pharmaceutical composition comprising a first anti-Bet v 1 antibody or antigen-binding fragment thereof and a second anti-Bet v 1 antibody or antigen-binding fragment thereof, wherein the first anti-Bet v 1 antibody comprises an HCDR1 comprising the amino acid sequence of SEQ ID NO:2, an HCDR2 comprising the amino acid sequence of SEQ ID NO:3, an HCDR3 comprising the amino acid sequence of SEQ ID NO:4, an LCDR1 comprising the amino acid sequence of SEQ ID NO: 6, an LCDR2 comprising the amino acid sequence of SEQ ID NO:7, and an LCDR3 comprising the amino acid sequence of SEQ ID NO:8; and wherein the second anti-Bet v 1 antibody comprises an HCDR1 comprising the amino acid sequence of SEQ ID NO:22, an HCDR2 comprising the amino acid sequence of SEQ ID NO:23, an HCDR3 comprising the amino acid sequence of SEQ ID NO:24, an LCDR1 comprising the amino acid sequence of SEQ ID NO:26, an LCDR2 comprising the amino acid sequence of SEQ ID NO:27, and an LCDR3 comprising the amino acid sequence of SEQ ID NO:28 (e.g., a pharmaceutical composition comprising the REGN5713 and REGN5715 anti-Bet v 1 antibodies), wherein the pharmaceutical composition does not comprise a third anti-Bet v 1 antibody. In some embodiments, the methods comprise administering to the subject one or more doses of the anti-Bet v 1 antibodies or of a cocktail consisting essentially of the REGN5713 and REGN5715 antibodies (e.g., a pharmaceutical composition consisting essentially of the REGN5713 and REGN5715 anti-Bet v 1 antibodies). In some embodiments, the methods comprise administering to the subject one or more doses of the anti-Bet v 1 antibodies or cocktail comprising the REGN5713 and REGN5715 antibodies (e.g., a pharmaceutical composition comprising the REGN5713 and REGN5715 anti-Bet v 1 antibodies) but excluding the REGN5714 anti-Bet v 1 antibody.

In some embodiments, a subject to be treated is sensitized to birch allergen, e.g., as measured by having a baseline serum allergen-specific IgE level ≥0.35 kUa/L for the allergen (e.g., birch tree pollen, Bet v 1 allergen, or Fagales allergen), or as measured by having a baseline positive skin prick test (SPT) with the allergen (e.g., a birch allergen SPT≥3 mm as compared to a negative control). In some embodiments, the subject to be treated has a baseline serum birch and Bet v 1 sIgE level ≥0.7 kUa/L. In some embodiments, the subject to be treated has a baseline positive SPT with an allergen (e.g., birch allergen extract or Fagales allergen), for example, a birch SPT of greater than or equal to 5 mm.

In some embodiments, a subject to be treated is sensitized to birch allergen and at least one birch-related allergen (including but not limited to alder, oak, and hazel), e.g., as measured by having a baseline serum allergen-specific IgE level ≥0.35 kUa/L for the allergen, or as measured by having a baseline positive SPT≥3 mM with the allergen. In some embodiments, a subject to be treated is sensitized to birch allergen and at least one birch-unrelated allergen (including but not limited to environmental allergens such as non-tree pollens, dust mite, cat dander, and dog dander), e.g., as measured by having a baseline serum allergen-specific IgE level ≥0.35 kUa/L for the allergen, or as measured by having a baseline positive SPT≥3 mM with the allergen.

In some embodiments, a subject to be treated has a baseline ocular itch score ≥2 in both eyes after a conjunctival allergen challenge (CAC). In some embodiments, the ocular itch score is measured using the Ora Calibra® Conjunctival Allergen Challenge Ocular Itching Scale. In some embodiments, a subject to be treated has a baseline conjunctival redness score ≥2 in both eyes after a CAC. In some embodiments, the conjunctival redness score is measured using the Ora Calibra® Ocular Hyperemia Scale.

In some embodiments, a subject to be treated has a history of birch tree pollen-triggered allergic rhinitis symptoms with or without conjunctivitis. In some embodiments, a subject to be treated has been diagnosed with a positive skin prick test (SPT) with a birch tree pollen extract. In some embodiments, the subject has a positive SPT with a mean wheal diameter ≥5 mm greater than a negative control. In some embodiments, a subject to be treated has been diagnosed with a positive allergen-specific IgE test for birch tree pollen (e.g., birch pollen extract) and/or a Bet v 1 antigen of ≥0.7 kUa/L.

In some embodiments, a subject to be treated has a history of moderate to severe birch pollen allergy. In some embodiments, the subject has a history of moderate to severe birch pollen allergy for at least 2 prior birch seasons.

In some embodiments, a subject to be treated is an adult. In some embodiments, the subject has a concomitant disease or condition. Non-limiting examples of concomitant diseases or conditions include allergy (e.g., allergy to one or more food allergens and/or allergy to one or more non-food allergens such as aeroallergens), oral allergy syndrome, and asthma. In some embodiments, the subject has asthma. In some embodiments, the subject has birch triggered asthma. In some embodiments, the subject has an allergy to birch allergen and one or more tree homologues (e.g., a Fagales allergen).

In some embodiments, a subject to be treated has a history of birch tree pollen-triggered allergic rhinitis symptoms with or without asthma. In some embodiments, a subject to be treated has a history of birch tree pollen-triggered allergic rhinitis symptoms with or without conjunctivitis with or without asthma. In some embodiments, a subject to be treated has a history of birch tree pollen-triggered allergic conjunctivitis.

In some embodiments, a subject is selected for treatment according to one or more of the following criteria:
Documented or participant-reported history of birch tree pollen-triggered allergic-rhinitis (AR) symptoms with or without conjunctivitis (for at least 2 seasons);
Positive Skin prick test (SPT) with birch tree pollen extract (mean wheal diameter at least 5 mm greater than a negative control);
Positive allergen-specific immunoglobulin E (slgE) tests for birch tree pollen and Bet v 1 (≥0.7 kUa/L); and/or.
Demonstrated TNSS≥6 out of 12 on at least 2 time points during the birch EEU exposure challenge.

In some embodiments, a subject is selected for treatment according to one or more of the following criteria:
Documented or participant-reported history of moderate to severe birch pollen allergy for at least 2 years with bothersome ocular symptoms during the birch season;
Positive SPT to birch allergen extract (mean wheal diameter at least 5 mm greater than the negative control);
Positive slgE tests for birch and Bet v 1 (both ≥0.7 kUa/L) at screening visit 1;
Meets the following criteria as defined below to confirm moderate to severe birch induced allergic conjunctivitis: (a) Bilateral positive CAC reactions* within approximately 10 minutes of birch allergen instillation during the birch screening titration CAC, AND (b) Bilateral positive CAC reactions in at least 2 out of 3 post-CAC time points, following instillation of the birch eliciting allergen dose (see Section 5.1), assessed during the birch confirmatory CAC. *Bilateral positive CAC reactions are defined as ocular itching ≥ 2/4 and conjunctival redness ≥ 2/4 in both eyes; and/or
Has a calculated best-corrected visual acuity of 0.7 logarithm of the minimum angle of resolution (Log MAR) (equivalent to 6/30 Snellen equivalence, or 20/100 vision) or better in each eye as measured using an ETDRS chart.

In some embodiments, treatment with the REGN5713/REGN5715 anti-Bet v 1 antibodies (or cocktail thereof) as disclosed herein results in an improvement in one or more signs or symptoms of birch allergy or an improvement in a condition associated with birch allergy. In some embodiments, treatment with the REGN5713 and REGN5715 anti-Bet v 1 antibodies (or cocktail thereof) treats signs and symptoms of allergic rhinitis in a subject with birch allergy. In some embodiments, treatment with the REGN5713 and REGN5715 anti-Bet v 1 antibodies (or cocktail thereof) treats signs and symptoms of allergic conjunctivitis in a subject with birch allergy.

In some embodiments, an improvement in one or more signs or symptoms of birch allergy in a subject is evaluated using an efficacy assessment tool as disclosed herein, in which a baseline value for the subject is determined. In some embodiments, the baseline value for the subject is determined using an allergen challenge procedure, such as a nasal allergen challenge (NAC) or a conjunctival allergen challenge (CAC). Nasal allergen challenge and conjunctival allergen challenge procedures are known in the art; see, e.g., Gevaert et al., J Allergy Clin Immunol 2022, 149:189-199; Meier et al., Clin Ophthalmol 2018, 12:2617-2628.

In some embodiments, treatment according to the methods disclosed herein improves one or more symptoms of allergic rhinitis in a subject. As used herein, “improving allergic rhinitis symptoms” includes reducing the severity or duration of or eliminating one or more symptoms of allergic rhinitis in the subject, such as but not limited to sneezing, itching (of nose, eyes, ears, or palate), rhinorrhea, postnasal drip, congestion, anosmia, headache, earache, tearing, red eyes, eye swelling, and fatigue. In some embodiments, a reduction in allergic rhinitis symptoms is measured by Total Nasal Symptom Score (TNSS). TNSS is a patient-reported composite symptom assessment of congestion, itching, rhinorrhea and sneezing in which patient-assessed symptom scores are assigned for each category for a given time point, using a four point scale (0-3), where 0 indicates no symptoms, a score of 1 for mild symptoms that are easily tolerated, 2 for awareness of symptoms which are bothersome but tolerable and 3 is reserved for severe symptoms that are hard to tolerate and interfere with daily activity. TNSS is calculated by adding the score for each of the symptoms to a total out of 12. In some embodiments, a TNSS score is measured after nasal allergen challenge (NAC) with an allergen. In some embodiments, a baseline TNSS score is measured for a subject (e.g., during a screening visit prior to the start of treatment). In some embodiments, the subject to be treated has a baseline TNSS score of at least a 6 (in a range of 0 to 12) at ≥2 timepoints prior to the start of treatment.

In some embodiments, treatment results in an improvement in TNSS during birch pollen season (e.g., over at least 28, 57, 85, or 113 days during birch pollen season or during an entire birch pollen season) relative to a baseline or control value (e.g., a baseline TNSS score for a subject prior to the start of treatment). In some embodiments, treatment results in a decrease in TNSS of at least 10%, 15%, 20%, 25%, 30%, 35%, 40%, 45%, 50% or more relative to a baseline or control value. In some embodiments, treatment results in an improvement in TNSS during birch pollen season (e.g., over at least 28, 57, 85, or 113 days during birch pollen season or during an entire birch pollen season) relative to treatment with an anti-Bet v 1 triple antibody cocktail including the REGN5714 antibody.

In some embodiments, treatment results in an improvement in TNSS after NAC (e.g., with birch pollen extract), wherein the improvement comprises a reduction in score for one or more of (i) congestion, (ii) itching, (iii) rhinorrhea, or (iv) sneezing, and/or total TNSS score, relative to a baseline score for the subject. In some embodiments, treatment results in a decrease in TNSS of at least 10%, 15%, 20%, 25%, 30%, 35%, 40%, 45%, 50% or more relative to a baseline score for the subject. In some embodiments, treatment results in a decrease in TNSS score of 1, 2, 3, 4, 5 or more points relative to a baseline score for the subject. In some embodiments, treatment results in a decrease in TNSS score relative to treatment with an anti-Bet v 1 triple antibody cocktail including the REGN5714 antibody.

In some embodiments, treatment according to the methods disclosed herein (e.g., administering the REGN5713 and REGN5715 anti-Bet v 1 antibodies or the REGN5713 and REGN5715 anti-Bet v 1 antibody cocktail as disclosed herein, wherein the method does not comprise administering a third anti-Bet v 1 antibody to the subject) reduces a subject's TNSS AUC (0-1 hr) by at least about 15%, 20%, 25%, 30%, 35% or more relative to a baseline TNSS AUC (0-1 hr) for the subject (e.g., a baseline TNSS AUC (0-1 hr) for the subject prior to the onset of treatment) and/or reduces TNSS AUC (0-1 hr) relative to treatment with an anti-Bet v 1 triple antibody cocktail including the REGN5714 antibody. In some embodiments, the TNSS AUC (0-1 hr) is measured after NAC. In some embodiments, treatment reduces a subject's peak TNSS by at least about 15%, 20%, 25%, 30%, 35% or more relative to a baseline peak TNSS for the subject (e.g., a baseline peak TNSS for the subject prior to the onset of treatment). In some embodiments, treatment reduces a subject's peak TNSS relative to treatment with an anti-Bet v 1 triple antibody cocktail including the REGN5714 antibody (e.g., a baseline peak TNSS for the subject prior to the onset of treatment). In some embodiments, the peak TNSS is measured after NAC. In some embodiments, the baseline peak TNSS is evaluated by determining the dose of allergen (e.g., Bet v 1 allergen or birch extract) that achieves TNSS of ≥7 in the subject prior to the onset of treatment, and the peak TNSS after treatment is evaluated by administering to the subject the same dose of allergen that achieved TNSS≥7 at baseline.

In some embodiments, treatment according to the methods disclosed herein improves one or more signs or symptoms of allergic conjunctivitis in a subject. As used herein, “improving signs or symptoms of allergic conjunctivitis” includes reducing the severity or duration of or eliminating one or more signs or symptoms of allergic conjunctivitis in the subject, such as but not limited to itchy, red, tearing, or puffy eyes. In some embodiments, a reduction in allergic conjunctivitis symptoms is measured by ocular itch score, conjunctival redness score, ciliary redness score, episcleral redness score, total redness score, tearing score, TOSS, chemosis score, or eyelid swelling score.

In some embodiments, a reduction in allergic conjunctivitis symptoms is measured by ocular itch score. In some embodiments, an ocular itch score is measured using the Ora Calibra® Conjunctival Allergen Challenge Ocular Itching Scale (0 to 4 with 0.5 unit increments). In some embodiments, an ocular itch score is measured after an allergen challenge (e.g., CAC). In some embodiments, a baseline ocular itch score is measured for a subject (e.g., during a screening visit prior to the start of treatment). In some embodiments, treatment results in an improvement in ocular itch score of at least 10%, 15%, 20%, 25%, 30%, 35%, 40%, 45%, 50% or more relative to a baseline score for the subject.

In some embodiments, a reduction in allergic conjunctivitis symptoms is measured by an ocular redness score. In some embodiments, ocular redness (comprising conjunctival redness, ciliary redness, and episcleral redness) is measured using the Ora Calibra® Ocular Hyperemia Scale (for conjunctival, ciliary, and episcleral vessel beds using a slit lamp: 0 to 4 with 0.5 unit increments). A total redness score is calculated as a sum of the bilateral averages for conjunctival redness score+ciliary redness score+episcleral redness score (range=0 to 12). In some embodiments, an ocular redness score is measured after an allergen challenge (e.g., CAC). In some embodiments, a baseline ocular redness score (e.g., conjunctival redness, ciliary redness, episcleral redness, or total redness) is measured for a subject (e.g., during a screening visit prior to the start of treatment). In some embodiments, treatment results in an improvement in total redness score, or one or more of the score components of conjunctival redness, ciliary redness, and episcleral redness, of at least 10%, 15%, 20%, 25%, 30%, 35%, 40%, 45%, 50% or more relative to a baseline score for the subject.

In some embodiments, a reduction in allergic conjunctivitis symptoms is measured by a tearing score. In some embodiments, a tearing score is measured using the Ora Calibra® Conjunctival Allergen Challenge Tearing Scale (0 to 4 with 1 unit increments). In some embodiments, a tearing score is measured after an allergen challenge (e.g., CAC). In some embodiments, a baseline tearing score is measured for a subject (e.g., during a screening visit prior to the start of treatment). In some embodiments, treatment results in an improvement in tearing score of at least 10%, 15%, 20%, 25%, 30%, 35%, 40%, 45%, 50% or more relative to a baseline score for the subject.

In some embodiments, a reduction in allergic conjunctivitis symptoms is measured by Total Ocular Symptom Score (TOSS). TOSS is a patient-reported composite symptom assessment of ocular symptoms. In one embodiments, a TOSS score ranges from 0-6 and is based on two symptoms: itching/redness/gritty feeling and tearing/watering; each of the 2 symptoms is graded by the patient as 0 (absent), 1 (mild), 2 (moderate), or 3 (severe). In another embodiment, a TOSS score ranges from 0-12 and is based on four items: itching/burning, redness, watering and tearing, and puffiness and swelling; patient-assessed symptom scores are assigned for each category for a given time point, using a four point scale (0-3), where 0 indicates no symptoms, a score of 1 for mild symptoms that are easily tolerated, 2 for awareness of symptoms which are bothersome but tolerable and 3 is reserved for severe symptoms that are hard to tolerate and interfere with daily activity. In yet another embodiment, a TOSS score ranges from 0-12 and is based on three items: (i) an ocular itch score measured using the Ora Calibra® Conjunctival Allergen Challenge Ocular Itching Scale (graded 0 to 4); (ii) a conjunctival redness score measured using the Ora Calibra® Ocular Hyperemia Scale (graded 0 to 4); and (iii) a tearing score measured using the Ora Calibra® Conjunctival Allergen Challenge Tearing Scale (graded 0 to 4).

In some embodiments, a TOSS score is measured after allergen challenge (e.g., NAC or CAC). In some embodiments, a baseline TOSS score is measured for a subject (e.g., during a screening visit prior to the start of treatment). In some embodiments, treatment results in an improvement in TOSS after NAC (e.g., with birch pollen extract), wherein the improvement comprises a reduction in score for one or more of (i) itching/burning, (ii) redness, (iii) watering and tearing, or (iv) puffiness and swelling, and/or total TOSS score, relative to a baseline score for the subject. In some embodiments, treatment results in an improvement in TOSS after CAC (e.g., with birch allergen), wherein the improvement comprises a reduction in score for one or more of (i) ocular itch, (ii) conjunctival redness, and (iii) tearing, and/or total TOSS score, relative to a baseline score for the subject.

In some embodiments, treatment results in a decrease in TOSS of at least 10%, 15%, 20%, 25%, 30%, 35%, 40%, 45%, 50% or more relative to a baseline score for the subject. In some embodiments, treatment results in a decrease in TOSS score of 1, 2, 3, 4, 5 or more points relative to a baseline score for the subject. In some embodiments, treatment reduces a subject's TOSS AUC (0-1 hr) by at least about 15%, 20%, 25%, 30%, 35% or more relative to a baseline TOSS AUC (0-1 hr) for subject (e.g., a baseline TOSS AUC (0-1 hr) for the subject prior to the onset of treatment). In some embodiments, treatment results in a decrease in TOSS of at least 10% or more relative to treatment with a three-way anti-Bet v 1 cocktail including the REGN5714 antibody. In some embodiments, treatment results in a decrease in TOSS score of 1 or more points relative to treatment with a three-way anti-Bet v 1 cocktail including the REGN5714 antibody. In some embodiments, the TOSS AUC (0-1 hr) is measured after NAC.

In some embodiments, treatment according to the methods disclosed herein results in an improvement (i.e., reduction) in a subject's Total Symptom Score (TSS). TSS is calculated by adding together a subject's TNSS (ranging from 0-12) and TOSS (ranging from 0-6), for a combined TNSS of 0 to 18. In some embodiments, a baseline TSS score is measured for a subject (e.g., during a screening visit prior to the start of treatment). In some embodiments, treatment results in an improvement in TSS during birch pollen season (e.g., over at least 28, 57, 85, or 113 days during birch pollen season or during an entire birch pollen season) relative to a baseline or control value (e.g., a baseline TSS score for a subject prior to the start of treatment). In some embodiments, treatment results in a decrease in TSS of at least 10%, 15%, 20%, 25%, 30%, 35%, 40%, 45%, 50% or more relative to a baseline or control value. In some embodiments, treatment results in a decrease in TSS score of 1, 2, 3, 4, 5 or more points relative to a baseline score for the subject. In some embodiments, treatment results in a decrease in TSS of at least 10% or more relative to treatment with an anti-Bet v 1 triple antibody cocktail including the REGN5714 antibody. In some embodiments, treatment results in a decrease in TSS score of 1 or more points relative to treatment with an anti-Bet v 1 triple antibody cocktail including the REGN5714 antibody.

In some embodiments, a reduction in allergic conjunctivitis symptoms is measured by chemosis score. In some embodiments, a chemosis score is measured using the Ora Calibra® Chemosis Scale (0 to 4 with 0.5 unit increments). In some embodiments, a chemosis score is measured after an allergen challenge (e.g., CAC). In some embodiments, a baseline chemosis score is measured for a subject (e.g., during a screening visit prior to the start of treatment). In some embodiments, treatment results in an improvement in chemosis score of at least 10%, 15%, 20%, 25%, 30%, 35%, 40%, 45%, 50% or more relative to a baseline score for the subject.

In some embodiments, a reduction in allergic conjunctivitis symptoms is measured by eyelid swelling score. In some embodiments, an eyelid swelling score is measured using the Ora Calibra® Conjunctival Allergen Challenge Eyelid Swelling Scale (0 to 3 with 1 unit increments). In some embodiments, an eyelid swelling score is measured after an allergen challenge (e.g., CAC). In some embodiments, a baseline eyelid swelling score is measured for a subject (e.g., during a screening visit prior to the start of treatment). In some embodiments, treatment results in an improvement in eyelid swelling score of at least 10%, 15%, 20%, 25%, 30%, 35%, 40%, 45%, 50% or more relative to a baseline score for the subject.

In some embodiments, treatment according to the methods disclosed herein results in an improvement (i.e., reduction) in a subject's Daily Medication Score (DMS). For calculating a DMS, a subject records their daily rescue medication use, including which medication(s) and the amount of these pre-specified medication(s). This information is used to calculate the DMS as follows: desloratadine 5 mg 6 points/dose; maximum daily score 6 points, olopatadine 1 mg/ml each drop 1.5 points/drop; maximum daily score 6 points, mometasone furoate 50 μg/dose 2.0 points/spray; maximum daily score 8 points). The maximum DMS score is 20. See, Calderon et al., Clin Exp Allergy 2014; 44 (10): 1228-39. In some embodiments, a baseline DMS score is measured for a subject (e.g., during a screening visit prior to the start of treatment). In some embodiments, treatment results in an improvement in DMS during birch pollen season (e.g., over at least 28, 57, 85, or 113 days during birch pollen season or during an entire birch pollen season) relative to a baseline or control value (e.g., a baseline TSS score for a subject prior to the start of treatment). In some embodiments, treatment results in an improvement in DMS during birch pollen season (e.g., over at least 28, 57, 85, or 113 days during birch pollen season or during an entire birch pollen season) relative to treatment with an anti-Bet v 1 triple antibody cocktail including the REGN5714 antibody. In some embodiments, treatment results in a decrease in DMS of at least 10%, 15%, 20%, 25%, 30%, 35%, 40%, 45%, 50% or more relative to a baseline or control value. In some embodiments, treatment results in a decrease in DMS score of 1, 2, 3, 4, 5 or more points relative to a baseline score for the subject.

In some embodiments, treatment according to the methods disclosed herein results in an improvement (i.e., reduction) in a subject's combined symptom and medication score (CSMS). CSMS is calculated by adding together a subject's DMS (ranging from 0-20) and TSS (ranging from 0-18), for a combined CSMS of 0 to 38. In some embodiments, a baseline CSMS score is measured for a subject (e.g., during a screening visit prior to the start of treatment). In some embodiments, treatment results in an improvement in CSMS during birch pollen season (e.g., over at least 28, 57, 85, or 113 days during birch pollen season or during an entire birch pollen season) relative to a baseline or control value (e.g., a baseline CSMS score for a subject prior to the start of treatment). In some embodiments, treatment results in a decrease in CSMS of at least 10%, 15%, 20%, 25%, 30%, 35%, 40%, 45%, 50% or more relative to a baseline or control value. In some embodiments, treatment results in a decrease in CSMS score of 1, 2, 3, 4, 5, 6, 7, 8, 9, 10 or more points relative to a baseline score for the subject. In some embodiments, treatment results in an improvement in CSMS during birch pollen season (e.g., over at least 28, 57, 85, or 113 days during birch pollen season or during an entire birch pollen season) relative to treatment with an anti-Bet v 1 triple antibody cocktail including the REGN5714 antibody.

In some embodiments, treatment according to the methods disclosed herein improves a subject's peak nasal inspiratory flow (PNIF) as compared to a baseline value (e.g., a baseline PNIF for the subject prior to the onset of treatment). In some embodiments, the PNIF is measured after NAC. In some embodiments, treatment increases a subject's PNIF by at least about 15%, 20%, 25%, 30%, 35%, 40%, 45%, 50%, or more relative to a baseline PNIF for the subject (e.g., a baseline PNIF for the subject prior to the onset of treatment). In some embodiments, treatment according to the methods disclosed herein improves a subject's peak nasal inspiratory flow (PNIF) relative to treatment with an anti-Bet v 1 triple antibody cocktail including the REGN5714 antibody.

In some embodiments, treatment according to the methods disclosed herein reduces a subject's allergen sensitization (e.g., sensitization to birch allergen) as compared to a baseline value (e.g., the subject's level of sensitization prior to the onset of treatment). In some embodiments, treatment reduces a subject's allergen sensitization (e.g., birch sensitization) by at least about 20%, 25%, 30%, 35%, 40%, 45%, 50%, 55%, 60%, 65%, 70%, 75% or more as compared to the subject's level of sensitization prior to the onset of treatment. In some embodiments, level of sensitization is measured using a skin prick test with the allergen (e.g., birch allergen extract). In some embodiments, level of sensitization is assessed by measuring serum antibodies (e.g., allergen specific IgE levels, such as Bet v 1 or birch pollen IgE). In some embodiments, treatment according to the methods disclosed herein reduces a subject's allergen sensitization (e.g., sensitization to birch allergen) relative to treatment with an anti-Bet v 1 triple antibody cocktail including the REGN5714 antibody.

In some embodiments, treatment according to the methods disclosed herein results in an increase in the subject's number of well days during birch pollen season. As used herein, a “well day” is defined as a day when the subject's TSS is ≤2 without the use of anti-allergy rescue medication.

In some embodiments, treatment according to the methods disclosed herein improves one or more symptoms of oral allergy syndrome. Oral allergy syndrome symptoms typically include itching of lips, mouth, and throat, and can also include lip and tongue swelling and angioedema. In some embodiments, methods of treating birch pollen-related oral allergy syndrome by administering the anti-Bet v 1 antibodies or anti-Bet v 1 antibody cocktail as disclosed herein are provided.

In some embodiments, treatment according to the methods disclosed herein results in one or more improvements as described above for a prolonged period of time, e.g., for at least one month, at least two months, at least three months, at least four months, or longer.

### Anti-Bet v 1 Antibodies and Antigen-Binding Fragments Thereof

In some embodiments, the first anti-Bet v 1 antibody or antigen-binding fragment thereof comprises the heavy chain complementarity determining regions (HCDRs) of a heavy chain variable region (HCVR) comprising the amino acid sequence of SEQ ID NO:1 and the light chain complementarity determining regions (LCDRs) of a light chain variable region (LCVR) comprising the amino acid sequence of SEQ ID NO:5. In some embodiments, the anti-Bet v 1 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO: 3, the HCDR3 comprises the amino acid sequence of SEQ ID NO:4, the LCDR1 comprises the amino acid sequence of SEQ ID NO:6, the LCDR2 comprises the amino acid sequence of SEQ ID NO:7, and the LCDR3 comprises the amino acid sequence of SEQ ID NO: 8. In some embodiments, the anti-Bet v 1 antibody or antigen-binding fragment thereof comprises the HCDR1, HCDR2, HCDR3, LCDR1, LCDR2, and LCDR3 of SEQ ID NOs: 2, 3, 4, 6, 7, and 8, respectively, and further comprises an HCVR having at least 85% sequence identity (e.g., at least 90%, 91%, 92%, 93%, 94%, 95%, 96%, 97%, 98%, or 99% sequence identity) to the amino acid sequence of SEQ ID NO:1 and an LCVR having at least 85% sequence identity (e.g., at least 90%, 91%, 92%, 93%, 94%, 95%, 96%, 97%, 98%, or 99% sequence identity) to the amino acid sequence of SEQ ID NO:5. In some embodiments, the anti-Bet v 1 antibody or antigen-binding fragment thereof comprises an HCVR comprising SEQ ID NO:1 and an LCVR comprising SEQ ID NO:5. In some embodiments, the anti-Bet v 1 antibody or antigen-binding fragment thereof comprises a heavy chain comprising the amino acid sequence of SEQ ID NO: 9. In some embodiments, the anti-Bet v 1 antibody or antigen-binding fragment thereof comprises a light chain comprising the amino acid sequence of SEQ ID NO:10. In some embodiments, the anti-Bet v 1 antibody is REGN5713, also known as bremzalerbart.

In some embodiments, the second anti-Bet v 1 antibody or antigen-binding fragment thereof comprises the HCDRs of a HCVR comprising the amino acid sequence of SEQ ID NO:21 and the LCDRs of a LCVR comprising the amino acid sequence of SEQ ID NO: 25. In some embodiments, the anti-Bet v 1 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:22, the HCDR2 comprises the amino acid sequence of SEQ ID NO:23, the HCDR3 comprises the amino acid sequence of SEQ ID NO:24, the LCDR1 comprises the amino acid sequence of SEQ ID NO:26, the LCDR2 comprises the amino acid sequence of SEQ ID NO:27, and the LCDR3 comprises the amino acid sequence of SEQ ID NO:28. In some embodiments, the anti-Bet v 1 antibody or antigen-binding fragment thereof comprises the HCDR1, HCDR2, HCDR3, LCDR1, LCDR2, and LCDR3 of SEQ ID NOs: 22, 23, 24, 26, 27, and 28, respectively, and further comprises an HCVR having at least 85% sequence identity (e.g., at least 90%, 91%, 92%, 93%, 94%, 95%, 96%, 97%, 98%, or 99% sequence identity) to the amino acid sequence of SEQ ID NO:21 and an LCVR having at least 85% sequence identity (e.g., at least 90%, 91%, 92%, 93%, 94%, 95%, 96%, 97%, 98%, or 99% sequence identity) to the amino acid sequence of SEQ ID NO:25. In some embodiments, the anti-Bet v 1 antibody or antigen-binding fragment thereof comprises an HCVR comprising SEQ ID NO:21 and an LCVR comprising SEQ ID NO: 25. In some embodiments, the anti-Bet v 1 antibody or antigen-binding fragment thereof comprises a heavy chain comprising the amino acid sequence of SEQ ID NO:29. In some embodiments, the anti-Bet v 1 antibody or antigen-binding fragment thereof comprises a light chain comprising the amino acid sequence of SEQ ID NO:30. In some embodiments, the anti-Bet v 1 antibody is REGN5715, also known as atisnolerbart.

In some embodiments, the anti-Bet v 1 antibody is a bioequivalent of an antibody disclosed herein (e.g., a bioequivalent of REGN5713 or REGN5715). The term “bioequivalent,” as used herein, refers to an anti-Bet v 1 antibody that is a pharmaceutical equivalent or pharmaceutical alternative whose rate and/or extent of absorption does not show a significant difference with that of the reference antibody (e.g., REGN5713 or REGN5715) when administered at the same molar dose under similar experimental conditions, either single dose or multiple dose. In some embodiments, the term refers to anti-Bet v 1 antibodies which do not have clinically meaningful differences with an anti-Bet v 1 antibody of the present disclosure (e.g., REGN5713 or REGN5715) in their safety, purity and/or potency.

In some embodiments, the anti-Bet v 1 antibody is an IgG1 or an IgG4 antibody. In some embodiments, the anti-Bet v 1 antibody comprises a heavy chain constant region of a human IgG1 or IgG4 isotype in which the constant region comprises one or more amino acid modifications (e.g., substitutions or deletions), e.g., an amino acid modification in the hinge, C_{H}2, or C_{H}3 region.

In some embodiments, an anti-Bet v 1 antibody used in the methods of the present disclosure can have pH-dependent binding characteristics. For example, an anti-Bet v 1 antibody for use in the methods of the present disclosure may exhibit reduced binding to Bet v 1 at acidic pH as compared to neutral pH. Alternatively, an anti-Bet v 1 antibody of the disclosure may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

In some embodiments, the therapeutic methods disclosed herein comprise the use of two anti-Bet v 1 antibodies as disclosed herein, e.g., a pharmaceutical composition comprising the REGN5713 and REGN5715 anti-Bet v 1 antibodies as disclosed herein. In some embodiments, the therapeutic methods disclosed herein comprise the use of two anti-Bet v 1 antibodies as disclosed herein, e.g., a pharmaceutical composition consisting essentially of the REGN5713 and REGN5715 anti-Bet v 1 antibodies as disclosed herein. In some embodiments, the therapeutic methods disclosed herein comprise the use of two anti-Bet v 1 antibodies as disclosed herein, e.g., a pharmaceutical composition comprising the REGN5713 and REGN5715 anti-Bet v 1 antibodies and excluding the REGN5714 antibody as disclosed herein.

In some embodiments, the combination or the pharmaceutical composition comprises:
(a) a first anti-Bet v 1 antibody or antigen-binding fragment thereof, wherein the first anti-Bet v 1 antibody comprises a heavy chain complementarity determining region (HCDR) 1 comprising the amino acid sequence of SEQ ID NO:2, an HCDR2 comprising the amino acid sequence of SEQ ID NO:3, an HCDR3 comprising the amino acid sequence of SEQ ID NO:4, a light chain complementarity determining region (LCDR) 1 comprising the amino acid sequence of SEQ ID NO:6, an LCDR2 comprising the amino acid sequence of SEQ ID NO:7, and an LCDR3 comprising the amino acid sequence of SEQ ID NO:8; and
(b) a second anti-Bet v 1 antibody or antigen-binding fragment thereof, wherein the second anti-Bet v 1 antibody comprises an HCDR1 comprising the amino acid sequence of SEQ ID NO: 22, an HCDR2 comprising the amino acid sequence of SEQ ID NO:23, an HCDR3 comprising the amino acid sequence of SEQ ID NO:24, an LCDR1 comprising the amino acid sequence of SEQ ID NO:26, an LCDR2 comprising the amino acid sequence of SEQ ID NO:27, and an LCDR3 comprising the amino acid sequence of SEQ ID NO:28.

In some embodiments, the combination or the pharmaceutical composition consists essentially of:
(a) a first anti-Bet v 1 antibody or antigen-binding fragment thereof, wherein the first anti-Bet v 1 antibody comprises a heavy chain complementarity determining region (HCDR) 1 comprising the amino acid sequence of SEQ ID NO:2, an HCDR2 comprising the amino acid sequence of SEQ ID NO:3, an HCDR3 comprising the amino acid sequence of SEQ ID NO:4, a light chain complementarity determining region (LCDR) 1 comprising the amino acid sequence of SEQ ID NO:6, an LCDR2 comprising the amino acid sequence of SEQ ID NO:7, and an LCDR3 comprising the amino acid sequence of SEQ ID NO:8; and
(b) a second anti-Bet v 1 antibody or antigen-binding fragment thereof, wherein the second anti-Bet v 1 antibody comprises an HCDR1 comprising the amino acid sequence of SEQ ID NO: 22, an HCDR2 comprising the amino acid sequence of SEQ ID NO:23, an HCDR3 comprising the amino acid sequence of SEQ ID NO:24, an LCDR1 comprising the amino acid sequence of SEQ ID NO:26, an LCDR2 comprising the amino acid sequence of SEQ ID NO:27, and an LCDR3 comprising the amino acid sequence of SEQ ID NO:28.

In some embodiments, the combination or the pharmaceutical composition comprises:
(a) a first anti-Bet v 1 antibody or antigen-binding fragment thereof, wherein the first anti-Bet v 1 antibody comprises a heavy chain complementarity determining region (HCDR) 1 comprising the amino acid sequence of SEQ ID NO:2, an HCDR2 comprising the amino acid sequence of SEQ ID NO:3, an HCDR3 comprising the amino acid sequence of SEQ ID NO:4, a light chain complementarity determining region (LCDR) 1 comprising the amino acid sequence of SEQ ID NO:6, an LCDR2 comprising the amino acid sequence of SEQ ID NO:7, and an LCDR3 comprising the amino acid sequence of SEQ ID NO:8; and
(b) a second anti-Bet v 1 antibody or antigen-binding fragment thereof, wherein the second anti-Bet v 1 antibody comprises an HCDR1 comprising the amino acid sequence of SEQ ID NO: 22, an HCDR2 comprising the amino acid sequence of SEQ ID NO:23, an HCDR3 comprising the amino acid sequence of SEQ ID NO:24, an LCDR1 comprising the amino acid sequence of SEQ ID NO:26, an LCDR2 comprising the amino acid sequence of SEQ ID NO:27, and an LCDR3 comprising the amino acid sequence of SEQ ID NO:28; but excludes
an anti-Bet v 1 antibody or antigen-binding fragment thereof comprising an HCDR1 comprising the amino acid sequence of SEQ ID NO:12, an HCDR2 comprising the amino acid sequence of SEQ ID NO:13, an HCDR3 comprising the amino acid sequence of SEQ ID NO:14, an LCDR1 comprising the amino acid sequence of SEQ ID NO:16, an LCDR2 comprising the amino acid sequence of SEQ ID NO:17, and an LCDR3 comprising the amino acid sequence of SEQ ID NO:18, e.g., REGN5714.

### Preparation of Human Antibodies

Methods for generating human antibodies in transgenic mice are known in the art. Any such known methods can be used in the context of the present disclosure to make human antibodies that specifically bind to the Bet v 1 protein.

Using VELOCIMMUNE™ technology (see, for example, U.S. Pat. No. 6,596,541, Regeneron Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate the fully human antibody of the disclosure, for example wild-type or modified IgG1 or IgG4. While the constant region selected may vary according to specific use, high affinity antigen-binding and target specificity characteristics reside in the variable region.

In general, the antibodies that can be used in the methods of the present disclosure possess high affinities, as described above, when measured by binding to antigen either immobilized on solid phase or in solution phase. The mouse constant regions are replaced with desired human constant regions to generate the fully human antibodies of the disclosure. While the constant region selected may vary according to specific use, high affinity antigen-binding and target specificity characteristics reside in the variable region.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

### Pharmaceutical Compositions

In one aspect, the present disclosure provides methods that comprise administering the first and second anti-Bet v 1 antibodies as disclosed herein (e.g., REGN5713 and REGN5715) to a subject, wherein the anti-Bet v 1 antibodies are independently or in combination contained within a pharmaceutical composition that comprises one or more pharmaceutically acceptable vehicle, carriers, and/or excipients. In some embodiments, the first and second anti-Bet v 1 antibodies are for use in treating birch allergy or for treating a condition associated with birch allergy (e.g., allergic rhinitis or oral allergy syndrome).

In some embodiments, the pharmaceutical composition comprises two anti-Bet v 1 antibodies or antigen-binding fragments thereof. For example, in some embodiments, the pharmaceutical composition comprises an antibody comprising the HCDR1, HCDR2, HCDR3, LCDR1, LCDR2, and LCDR3 of SEQ ID NOs: 2, 3, 4, 6, 7, and 8, respectively, and an antibody comprising the HCDR1, HCDR2, HCDR3, LCDR1, LCDR2, and LCDR3 of SEQ ID NOs: 22, 23, 24, 26, 27, and 28, respectively. In some embodiments, the pharmaceutical composition consists essentially of two anti-Bet v 1 antibodies or antigen-binding fragments thereof. For example, in some embodiments, the pharmaceutical composition consists essentially of an antibody comprising the HCDR1, HCDR2, HCDR3, LCDR1, LCDR2, and LCDR3 of SEQ ID NOs: 2, 3, 4, 6, 7, and 8, respectively, and an antibody comprising the HCDR1, HCDR2, HCDR3, LCDR1, LCDR2, and LCDR3 of SEQ ID NOs: 22, 23, 24, 26, 27, and 28, respectively.

In some embodiments, the pharmaceutical composition comprises two anti-Bet v 1 antibodies or antigen-binding fragments thereof and excludes the REGN5714 anti-Bet v 1 antibody. For example, in some embodiments, the pharmaceutical composition comprises an antibody comprising the HCDR1, HCDR2, HCDR3, LCDR1, LCDR2, and LCDR3 of SEQ ID NOs: 2, 3, 4, 6, 7, and 8, respectively, and an antibody comprising the HCDR1, HCDR2, HCDR3, LCDR1, LCDR2, and LCDR3 of SEQ ID NOs: 22, 23, 24, 26, 27, and 28, respectively, and excludes the REGN5714 anti-Bet v 1 antibody.

In some embodiments, the pharmaceutical composition comprises or consists essentially of each of the first anti-Bet v 1 antibody and the second anti-Bet v 1 antibody and excludes a third anti-Bet v 1 antibody. In some embodiments, the pharmaceutical composition comprises or consists essentially of each of the first anti-Bet v 1 antibody and the second anti-Bet v 1 antibody, and excludes a third anti-Bet v 1 antibody comprising an HCDR1 comprising the amino acid sequence of SEQ ID NO:12, an HCDR2 comprising the amino acid sequence of SEQ ID NO:13, an HCDR3 comprising the amino acid sequence of SEQ ID NO:14, an LCDR1 comprising the amino acid sequence of SEQ ID NO:16, an LCDR2 comprising the amino acid sequence of SEQ ID NO:17, and an LCDR3 comprising the amino acid sequence of SEQ ID NO: 18, i.e., REGN5714.

Various pharmaceutically acceptable carriers and excipients are well-known in the art. See, e.g., Remington's Pharmaceutical Sciences, Mack Publishing Company, Easton, PA. In some embodiments, the carrier is suitable for intravenous, intramuscular, oral, intraperitoneal, intrathecal, transdermal, topical, or subcutaneous administration.

The dose of the REGN5713 and REGN5715 anti-Bet v 1 antibodies that are administered to a patient according to the methods of the present disclosure may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-Bet v 1 antibodies may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351). Specific exemplary doses of anti-Bet v 1 antibodies, and administration regimens involving the same, that can be used in the context of the present disclosure are disclosed elsewhere herein.

Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously.

In some embodiments, a pharmaceutical composition of the present disclosure is contained within a container. Thus, in another aspect, containers comprising a pharmaceutical composition as disclosed herein are provided. For example, in some embodiments, a pharmaceutical composition is contained within a container selected from the group consisting of a glass vial, a syringe, a pen delivery device, and an autoinjector.

In some embodiments, a pharmaceutical composition of the present disclosure is delivered, e.g., subcutaneously or intravenously, with a standard needle and syringe. In some embodiments, the syringe is a pre-filled syringe. In some embodiments, a pen delivery device or autoinjector is used to deliver a pharmaceutical composition of the present disclosure (e.g., for subcutaneous delivery). A pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPENT, OPTIPEN PRO™, OPTIPEN STARLET™, and OPTICLIK™ (Sanofi-Aventis, Frankfurt, Germany). Examples of disposable pen delivery devices having applications in subcutaneous delivery of a pharmaceutical composition of the present disclosure include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park Ill.).

In some embodiments, the pharmaceutical composition is delivered using a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

In some embodiments, pharmaceutical compositions for use as described herein are prepared into dosage forms in a unit dose suited to fit a dose of the active ingredients. Such dosage forms in a unit dose include, for example, tablets, pills, capsules, injections (ampoules), suppositories, etc.

### Dosage and Administration Regimens

Typically, an amount of an anti-Bet v 1 antibody that is administered to a subject according to the methods disclosed herein is a therapeutically effective amount. As used herein, the phrase “therapeutically effective amount” means an amount of an anti-Bet v 1 antibody (or combination of anti-Bet v 1 antibodies) that results in one or more of: (a) a reduction in the severity or duration of one or more symptoms of birch allergy; (b) prevention or alleviation of an allergic reaction to a birch allergen (e.g., birch pollen extract or a Bet v 1 protein); (c) reduction in provoked allergic rhinitis symptoms after nasal allergen challenge; (d) reduction in the level of one or more markers of Type 2 immune activity (e.g., serum TARC or total IgE); and (e) a reduction in the use or need for conventional allergy therapy (e.g., reduced or eliminated use of antihistamines, decongestants, nasal or inhaled steroids, anti-IgE treatment, epinephrine, etc.).

In some embodiments, a therapeutically effective amount of each of the two anti-Bet v 1 antibodies as disclosed herein (e.g., each of REGN5713 and REGN5715) is administered to the subject. In some embodiments, each of the anti-Bet v 1 antibodies is administered in an amount from about 50 mg to about 600 mg, about 50 mg to about 450 mg, about 50 mg to about 300 mg, about 100 mg to about 400 mg, or about 100 mg to about 300 mg, e.g., about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg. In some embodiments, each of the two anti-Bet v 1 antibodies (e.g., REGN5713 and REGN5715) is administered in an amount of about 50 mg, 100 mg, 150 mg, 200 mg, 250 mg, 300 mg, or 350 mg. In some embodiments, the two anti-Bet v 1 antibodies are administered in the same amount. In some embodiments, the two anti-Bet v 1 antibodies are administered in different amounts.

In some embodiments, the anti-Bet v 1 antibodies (e.g., an antibody comprising the CDRs and/or HCVR and LCVR sequences of REGN5713 and/or REGN5715 as disclosed herein) are administered at a total dose of about 100 mg to about 1500 mg, e.g., about 100 mg to about 1000 mg, about 150 mg to about 1000 mg, about 200 mg to about 1000 mg, about 300 mg to about 1000 mg, about 200 mg to about 800 mg, or about 250 mg to about 750 mg. In some embodiments, the anti-Bet v 1 antibody or antibodies are administered at a total dose of about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1450 mg, about 1475 mg, or about 1500 mg.

In some embodiments, the REGN5713 and REGN5715 anti-Bet v 1 antibodies, or pharmaceutical composition comprising the two anti-Bet v 1 antibodies, are administered to a subject at a dosing frequency of about once every eight weeks, once every twelve weeks, or less frequently so long as a therapeutic response is achieved. In some embodiments, the anti-Bet v 1 antibodies are administered once every three months, once every four months, once every five months, once every six months, once every seven months, once every eight months, once every nine months, once every ten months, once every eleven months, or once every twelve months. In some embodiments, the anti-Bet v 1 antibodies are administered once a year or twice a year. In some embodiments, the anti-Bet v 1 antibodies are administered once a year or twice a year, prior to the onset of allergy season (e.g., prior to birch pollen season). The frequency of administration may also be adjusted during the course of treatment by a physician depending on the needs of the individual patient following clinical examination.

In some embodiments, the REGN5713 and REGN5715 anti-Bet v 1 antibodies, or pharmaceutical composition comprising the two anti-Bet v 1 antibodies, are administered to the subject as a single dose of each of the first anti-Bet v 1 antibody or antigen-binding fragment thereof and the second anti-Bet v 1 antibody or antigen-binding fragment thereof, or a pharmaceutical composition comprising the first and second anti-Bet v 1 antibodies or antigen-binding fragments thereof, prior to the start of birch pollen season. In some embodiments, the single dose is administered at least 1 week, 2 weeks, 3 weeks, or 4 weeks prior to the start of birch pollen season.

In certain embodiments involving the administration of the REGN5713 anti-Bet v 1 antibody and/or the REGN5715 anti-Bet v 1 antibody at a dosing frequency described herein, each dose is administered at an amount of about 50 mg to about 600 mg, e.g., about 50 mg, 100 mg, 150 mg, 200 mg, 250 mg, or 300 mg. In some aspects, for each dose that is administered, each of the two anti-Bet v 1 antibodies are administered in amount of about 50 mg to about 600 mg, e.g., about 50 mg, 100 mg, 150 mg, 200 mg, 250 mg, or 300 mg. In certain embodiments, for each dose that is administered, the two anti-Bet v 1 antibodies are administered in a 1:1 ratio. In some embodiments, the first anti-Bet v 1 antibody and second anti-Bet v 1 antibody are administered separately. In some embodiments, the first anti-Bet v 1 antibody and second anti-Bet v 1 antibody are co-administered. In some embodiments, the first anti-Bet v 1 antibody and second anti-Bet v 1 antibody are administered in a pharmaceutical composition, i.e., as a composition comprising the first and second anti-Bet v 1 antibodies.

In some embodiments, a pharmaceutical composition comprising each of a first anti-Bet v 1 antibody and a second anti-Bet v 1 antibody as disclosed herein is administered to a subject at a dose of about 300 mg per antibody (total dose for the two antibodies of about 600 mg). In some embodiments, a pharmaceutical composition consisting essentially of each of a first anti-Bet v 1 antibody and a second anti-Bet v 1 antibody as disclosed herein is administered to a subject at a dose of about 300 mg per antibody (total dose for the two antibodies of about 600 mg). In some embodiments, the pharmaceutical composition is administered to the subject subcutaneously or intravenously.

In some embodiments, a pharmaceutical composition comprising each of a first anti-Bet v 1 antibody and a second anti-Bet v 1 antibody as disclosed herein is administered to a subject at a dose of about 150 mg per antibody (total dose for the two antibodies of about 300 mg). In some embodiments, a pharmaceutical composition consisting essentially of each of a first anti-Bet v 1 antibody and a second anti-Bet v 1 antibody as disclosed herein is administered to a subject at a dose of about 150 mg per antibody (total dose for the two antibodies of about 300 mg). In some embodiments, the pharmaceutical composition is administered to the subject subcutaneously or intravenously.

In some embodiments, the two anti-Bet v 1 antibodies are in separate pharmaceutical compositions. In some embodiments, the pharmaceutical compositions are administered at the same time (e.g., by combining the compositions in a solution prior to administration). In some embodiments, the pharmaceutical compositions are administered separately (e.g., by sequential administration).

### Combination Therapies

In some embodiments, the methods of the present disclosure comprise administering to the subject one or more additional therapeutic agents in combination with the anti-Bet v 1 antibodies (e.g., REGN5713 and REGN5715) or cocktail of the anti-Bet v 1 antibodies as disclosed herein. As used herein, the expression “in combination with” means that the additional therapeutic agents are administered before, after, or concurrent with the anti-Bet v 1 antibodies or pharmaceutical composition comprising the anti-Bet v 1 antibodies. The term “in combination with” also includes sequential or concomitant administration of the anti-Bet v 1 antibodies and a second therapeutic agent or therapy, with the proviso that the second therapeutic agent or therapy excludes a further anti-Bet v 1 antibody or antigen-binding fragment thereof (e.g., excludes REGN5714).

In some embodiments, the additional therapeutic agent is a steroid, an antihistamine, a decongestant, an anti-IgE agent, or an agent that depletes plasma cells and/or B cells. In some embodiments, the additional therapeutic agent is a steroid (e.g., a corticosteroid, such as an inhaled corticosteroid (ICS)). In some embodiments, the additional therapeutic agent is an antihistamine (e.g., loratadine, fexofenadine, cetirizine, diphenhydramine, promethazine, carbinoxamine, desloratadine, hydroxyzine, levocetirizine, triprolidine, brompheniramine, or chlorpheniramine). In some embodiments, the additional therapeutic agent is a decongestant (e.g., pseudoephedrine or phenylephrine). In some embodiments, the additional therapeutic agent is an anti-IgE agent (e.g., omalizumab).

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

REGN5713 is a fully human anti-Bet v 1 antibody comprising the HCVR of SEQ ID NO: 1, the HCDR1 of SEQ ID NO:2, the HCDR2 of SEQ ID NO:3, the HCDR3 of SEQ ID NO:4, the LCVR of SEQ ID NO:5, the LCDR1 of SEQ ID NO:6, the LCDR2 of SEQ ID NO:7, and the LCDR3 of SEQ ID NO:8.

REGN5715 is a fully human anti-Bet v 1 antibody comprising the HCVR of SEQ ID NO: 21, the HCDR1 of SEQ ID NO:22, the HCDR2 of SEQ ID NO:23, the HCDR3 of SEQ ID NO: 24, the LCVR of SEQ ID NO:25, the LCDR1 of SEQ ID NO:26, the LCDR2 of SEQ ID NO: 27, and the LCDR3 of SEQ ID NO:28.

### Example 1: A Single Dose of REGN5713 and REGN5715 is Efficacious in Reducing Birch Pollen Induced Allergic Nasal Symptoms

### Study Design and Objectives

A two-part, Phase 2, randomized, double-blind, placebo-controlled study was conducted in birch-allergic patients to assess the efficacy of anti-Bet v 1 antibodies (NCT05430919). Efficacy in the reduction of allergic rhinitis and conjunctivitis symptoms as well as birch skin test reactivity was tested using assessments out-of-season (Part A: EEU challenges and titrated skin prick testing) and during the birch pollen season (Part B: EEU challenges, field assessments, and titrated skin prick testing).

The primary objective of the study was to assess the efficacy of a single dose of the anti-Bet v 1 monoclonal antibodies in the reduction of allergic nasal symptoms during an out-of-season birch allergen environmental exposure unit (EEU) challenge in participants receiving REGN5713-5714-5715 versus placebo, as measured by the mean of Total Nasal Symptom Score [TNSS (2 to 6 hours)] during a birch allergen EEU challenge at day 29.

Secondary objectives included assessing (i) the magnitude and duration of efficacy of a single dose of the anti-Bet v 1 monoclonal antibodies in the reduction of allergic symptoms in participants receiving the different combinations of the anti-Bet v 1 monoclonal antibodies, (ii) the efficacy of the anti-Bet v 1 monoclonal antibodies in the reduction of skin test reactivity from pre-treatment baseline in participants receiving the different combinations of the anti-Bet v 1 monoclonal antibodies, (iii) differences in the degree of reduction of allergic symptoms during the out-of-season birch allergen EEU challenges in participants receiving the different combinations of the anti-Bet v 1 monoclonal antibodies, and (iv) differences in the degree of reduction of skin test reactivity from pre-treatment baseline in participants receiving the different combinations of the anti-Bet v 1 monoclonal antibodies. Other secondary objectives included assessing the efficacy of a single dose of the anti-Bet v 1 monoclonal antibodies in the reduction of allergic symptoms during the out-of-season oak allergen EEU challenge in the subpopulation of oak allergic participants; assessing health-related quality of life during birch pollen season and peak birch pollen season; and evaluating safety and tolerability of the antibodies as compared to placebo following single administration (dose #1) and after repeat dosing (dose #2).

In Part A (out-of-season), study participants were randomized to 1 of 4 arms and received a dose of either REGN5713-5714-5715 (“3-mAb”), REGN5713-5715 (“2-mAb”), REGN5715 (“1-mAb”), or matching placebo. Part A assessed whether a dose of either a monotherapy or a combination(s) of anti-Bet v 1 monoclonal antibodies (REGN5715, REGN5713-5715, REGN5713-5714-5715) demonstrated a greater reduction in TNSS than placebo in a birch EEU. Primary efficacy was assessed during the out-of-season birch allergen EEU challenge on day 29 after receiving a dose of the study drug on day 1.

After completion of all Part A assessments outside of the birch pollen season, participants started Part B of the study prior to the start of the birch pollen season. In Part B, participants received a dose (dose #2) of the study drug (the same monoclonal antibody[ies] or placebo administered to that participant in Part A), administered ahead of the anticipated birch pollen season (determined using historical local pollen data), which was intended as a single dose for coverage of the entire birch season. Pollen counts were monitored during the study period. The start of the birch pollen season was defined as the first of 3 consecutive days with a pollen count of 10 grains/m³ or greater. The end of the birch pollen season was defined as the last day of the last occurrence of 3 consecutive days with a pollen count of 10 grains/m³ or greater. The peak birch pollen season was defined as the 15 consecutive days within the birch pollen season with the highest 15-day moving average pollen count.

### Key Inclusion Criteria

Subjects participating in the clinical trial satisfied one or more of the following criteria:
Documented or participant-reported history of birch tree pollen-triggered allergic-rhinitis (AR) symptoms with or without conjunctivitis (for at least 2 seasons)
Positive Skin prick test (SPT) with birch tree pollen extract (mean wheal diameter at least 5 mm greater than a negative control) in screening period
Positive allergen-specific immunoglobulin E (slgE) tests for birch tree pollen and Bet v 1 (≥0.7 kUa/L) in screening period
Demonstrated TNSS≥6 out of 12 on at least 2 time points during the birch EEU exposure challenge in screening period

### Key Exclusion Criteria

Subjects were excluded from the trial for any of the following reasons:
Participation in a prior REGN5713-5714-5715 clinical trial and received REGN5713-5714-5715 antibodies (receipt of placebo in a previous trial was allowed).
Significant rhinitis, sinusitis, significant and/or severe allergies not associated with the birch pollen season, or due to daily contact with other non-birch related allergens causing symptoms that are expected to coincide or potentially interfere with the study EEU assessments or with the birch pollen season, as assessed by the investigator
Participants who anticipate major changes in allergen exposure during the birch pollen season that are expected to coincide with study assessments or planned travel that is expected to interfere with the study assessments, as assessed by the investigator (e.g., anticipated travel during planned EEU sessions or birch pollen season).
Persistent chronic or recurring acute infection requiring treatment with antibiotics, antivirals, or antifungals, or any untreated respiratory infections within 4 weeks prior to screening visit 1. Patients may be re-evaluated for eligibility after resolution of symptoms and specified duration.
History of significant, recurrent sinusitis, defined as at least 3 episodes requiring antibiotic treatment per year for the last 2 years.
Abnormal lung function as judged by the investigator with Forced Expiratory Volume (FEV1)<75% of predicted at screening or randomization
A clinical history of moderate to severe asthma, uncontrolled asthma, global initiative for asthma [GINA] steps 3 to 5, history of life-threatening asthma, asthma exacerbations due to tree pollen allergy within 2 prior seasons, >2 asthma exacerbations requiring systemic steroids in past 12 months, asthma related emergency care or hospitalization within 12 months prior to screening, as defined in the protocol.
History of birch or other tree allergen immunotherapy in the 3 years prior to screening.
Use of anti-IgE or other biological therapy (including but not limited to anti IL-5, anti IL-4) that interferes with type 2 disease within 6 months prior to screening visit 1.
Allergen-specific immunotherapy with any allergen other than birch or other trees at screening.
History of clinically significant cardiovascular, respiratory, hepatic, renal, gastrointestinal, endocrine, hematological, psychiatric, or neurological disease,
Any malignancy within the past 5 years, except for basal cell or squamous epithelial cell carcinomas of the skin or carcinoma in situ of the cervix or anus that have been resected, with no evidence of local recurrence or metastatic disease for 3 years.

### Study Treatments

### Part A:

Subcutaneous administration of a dose of study drug (dose #1)
3-mAb cocktail REGN5713-5714-5715 900 mg (300 mg per mAb)
2-mAb cocktail REGN5713-5715 600 mg (300 mg per mAb) plus placebo that replaced REGN5714
1-mAb REGN5715 300 mg plus placebo that replaced REGN5713-5714
Matching placebo that replaced active drug

### Part B:

Subcutaneous administration of a dose of study drug (dose #2)
3-mAb cocktail REGN5713-5714-5715 900 mg (300 mg per mAb)
2-mAb cocktail REGN5713-5715 600 mg (300 mg per mAb) plus placebo that replaced REGN5714
1-mAb REGN5715 300 mg plus placebo that replaced REGN5713-5714
Matching placebo that replaced active drug

Participants received the same treatment in Part B that they were originally randomized to receive in Part A.

### Parts A and B:

Participants were randomized to receive placebo or 1 of 3 active treatment arms. In order to maintain blinding, all participants received three 2-mL SC injections.

For REGN5713-5714-5715 (300 mg/mAb): 1:1:1 ratio resulted in 50 mg/ml REGN5713, 50 mg/mL REGN5714 and 50 mg/mL REGN5715. Three 2.0 mL SC injections were administered for 900 mg total.

For REGN5713-5715 (300 mg/mAb): 1:1 ratio resulted in 75 mg/mL REGN5713, 75 mg/mL REGN5715; Two 2.0 mL SC injections were administered for 600 mg total. One 2.0 mL placebo injection was used to match volume.

For REGN5715 (300 mg/mAb): at 150 mg/mL REGN5715; One 2.0 mL SC injection were administered for 300 mg total. Two 2.0 mL placebo injections were used to match volume.

### Efficacy Procedures

Procedures for assessing efficacy are described below.

Total Nasal Symptom Score (TNSS): The TNSS ranges from 0 to 12 and is based on assessment of 4 nasal symptoms graded on a Likert scale ranging from 0 (none) to 3 (severe) for congestion, itching, and rhinorrhea, and for sneezing.

Total Ocular Symptom Score (TOSS): The TOSS ranges from 0 to 6 and is based on 2 symptoms: itching/redness/gritty feeling and tearing/watering. Each of the 2 symptoms is graded 0 (absent), 1 (mild), 2 (moderate), or 3 (severe).

Total Symptom Score (TSS): TSS is calculated by adding the TNSS and TOSS together, for a combined TSS of 0 to 18.

Daily Medication Score: Subjects were asked to record their daily rescue medication use using an e-diary, including which medications and the amount of these pre-specified medications. This information was used to calculate the DMS as follows: desloratadine 5 mg 6 points/dose; maximum daily score 6 points, olopatadine 1 mg/mL each drop 1.5 points/drop; maximum daily score 6 points, mometasone furoate 50 μg/dose 2.0 points/spray; maximum daily score 8 points). The maximum DMS score is 20.

Combined Symptom and Medication Score (CSMS): The CSMS is calculated by adding the DMS and TSS together, with scores ranging between 0 and 38.

Asthma Control Questionnaire (ACQ): The ACQ measures the adequacy of asthma control and change in asthma control that occurs spontaneously or as a result of treatment. The ACQ-5 is comprised of 5, patient-reported items that were rated by clinicians as the most important to evaluate control: (1) awakening at night due to symptoms, (2) morning symptoms, (3) limitation of daily activities, (4) shortness of breath, and (5) wheezing. The total score ranges from 0 to 6 with higher scores denoting less asthma control. A score of ≥1.5 is considered as uncontrolled asthma.

Standardized Rhinoconjunctivitis Quality of Life Questionnaire (RQLQ(S)): The RQLQ(S) has 28 questions in 7 domains: activity limitation, sleep problems, nose symptoms, eye symptoms, non-nose/eye symptoms, practical problems, and emotional function. There are 3 subject-specific questions in the activity domain that allow subjects to select 3 activities in which they are most limited by their rhinoconjunctivitis. Subjects recall how bothered they have been by their rhinoconjunctivitis during the previous week and respond to each question on a 7-point scale (0 [not impaired at all] to 6 [severely impaired]). The overall RQLQ(S) score is the mean of all 28 responses, and the individual domain scores are the means of the items in those domains.

Patient Global Impression of Severity (PGI-S): The PGI-S assesses the severity of seasonal allergy symptoms over the past 1 week. Symptom severity ranges from 0 (no symptoms), 1 (mild), 2 (moderate), and 3 (severe) symptoms.

### Part A Results

Birch allergic patients were randomized 1:1:1:1 to Bet v 1 antibodies (REGN5713/5714/5715 [3-mAb], REGN5713/5715 [2-mAb] or REGN5715 [1-mAb]) or placebo. Out-of-season birch challenges were conducted in an environmental exposure unit (EEU) at screening, days 29, 57, and 85 after a single dose of the Bet v 1 antibody/antibodies or placebo. The primary endpoint for Part A was total nasal symptom score (TNSS; Range 0-12; nasal congestion, itching, runny-nose, sneezing, each scored 0-3; averaged during 2-6 hours of the EEU challenges) at day 29 comparing REGN5713/REGN5715/REGN5715 versus placebo. The TNSS at day 29 for REGN5713/5715 versus placebo and for REGN5715 versus placebo was assessed as secondary endpoints.

As shown in Table 1, baseline demographic characteristics were comparable between placebo-treated patients and those who received Bet v 1 antibody/antibodies. Among the 217 patients with moderate-to-severe birch allergy who participated in the study, the majority were polysensitized (96.8% [birch (or birch-related) and any birch unrelated allergen]). In other words, the patients were sensitized to one or more unrelated allergens. All patients were sensitized to birch; additionally, the most common birch-related allergens were alder and red oak, while the most common non-tree pollens were grass and short ragweed.

In Part A, the primary and all key secondary endpoints were met for the 3-ab, 2-ab, and 1-ab arms versus placebo at day 29. As shown in Table 2 below, a single dose of REGN5713/REGN5715/REGN5715 (3-ab) significantly reduced the mean TNSS versus placebo at day 29, with a treatment difference of −2.31 and 34.4% reduction from placebo. Surprisingly, however, a single dose of REGN5713/5715 (2-ab) was even more efficacious than the 3-ab combination, with a treatment difference of −2.65 and 39.5% reduction from placebo. Furthermore, durable efficacy was seen after a single dose, with nominal statistical significance achieved at all timepoints for 3-mAb and 2-mAb versus placebo (day 57:3-mAb-1.83 [p=0.0017], 2-mAb-2.4 [p<0.0001]; day 85:3-mAb-1.54 [p=0.0148], 2-mAb-1.66 [p=0.0081]). Surprisingly, the REGN5713/5715 (2-ab) was more efficacious than REGN5713/REGN5715/REGN5715 (3-ab) even at days 57 and 85 versus placebo. Overall, a similar proportion of patients experienced treatment-emergent adverse events across all groups.

TABLE 1
Baseline Demographic Characteristics

| | | | | | | | REGN5713- | |
|---|---|---|---|---|---|---|---|---|
| | Placebo | | REGN5715 | | REGN5713-5715 | | 5714-5715 | |
| | (n = 55) | | 300 mg (n = 55) | | 600 mg (n = 52) | | 900 mg (n = 55) | |
| Age, years, mean (SD) | 43.8 | (13.7) | 40.1 | (11.2) | 40.3 | (10.9) | 42.6 | (12.6) |
| Female sex, n (%) | 30 | (54.5) | 27 | (49.1) | 22 | (42.3) | 34 | (61.8) |
| Ethnicity, n (%) | | | | | | | | |
| Not Hispanic or Latino | 49 | (89.1) | 47 | (85.5) | 46 | (88.5) | 49 | (89.1) |
| Hispanic or Latino | 5 | (9.1) | 5 | (9.1) | 6 | (11.5) | 5 | (9.1) |
| Race, n (%) | | | | | | | | |
| White | 39 | (70.9) | 43 | (78.2) | 37 | (71.2) | 36 | (65.5) |
| Black/African American | 6 | (10.9) | 5 | (9.1) | 5 | (9.6) | 5 | (9.1) |
| Asian | 7 | (12.7) | 6 | (10.9) | 8 | (15.4) | 12 | (21.8) |
| American Indian/ | 0 | | 1 | (1.8) | 0 | | 0 | |
| Alaska Native | | | | | | | | |
| Not reported | 2 | (3.6) | 0 | | 1 | (1.9) | 2 | (3.6) |
| Other | 1 | (1.8) | 0 | | 1 | (1.9) | 0 | |
| BMI, kg/m², mean (SD) | 28.7 | (6.1) | 28.1 | (6.2) | 28.7 | (5.0) | 27.2 | (5.2) |
| Sensitized to allergens | | | | | | | | |
| at screening, n (%)^{a} | | | | | | | | |
| Birch-related | | | | | | | | |
| Birch | 55 | (100) | 55 | (100) | 52 | (100) | 55 | (100) |
| Hazel | 47 | (85.5) | 50 | (90.9) | 47 | (90.4) | 52 | (94.5) |
| Alder | 54 | (98.2) | 55 | (100) | 52 | (100) | 55 | (100) |
| Oak | 51 | (92.7) | 50 | (90.9) | 48 | (92.3) | 49 | (89.1) |
| Birch-unrelated | | | | | | | | |
| Short ragweed | 49 | (89.1) | 45 | (81.1) | 44 | (84.6) | 43 | (78.2) |
| Grass | 42 | (76.4) | 43 | (78.2) | 42 | (80.8) | 43 | (78.2) |
| House dust mite | 28 | (50.9) | 28 | (50.9) | 33 | (63.5) | 27 | (49.1) |
| Cat | 29 | (52.7) | 34 | (61.8) | 33 | (63.5) | 31 | (56.4) |
| Dog | 5 | (9.1) | 8 | (14.5) | 5 | (9.6) | 6 | (10.9) |
| Birch SPT mean wheal | 12.9 | (4.5) | 12.3 | (3.5) | 12.1 | (3.8) | 12.8 | (3.9) |
| diameter, mm, mean (SD) | | | | | | | | |
| Birch sIgE, kUa/L, | 15.5 | (24.8) | 12.5 | (19.0) | 10.2 | (14.6) | 15.0 | (29.8) |
| mean (SD) | | | | | | | | |
| Bet v1 sIgE, kUa/L, | 13.5 | (20.0) | 9.6 | (14.0) | 9.1 | (14.8) | 11.2 | (16.3) |
| mean (SD) | | | | | | | | |

^{a}Sensitized to other allergens by SPT or sIgE defined as SPT ≥3 mm relative to negative control or sIgE ≥0.35 kUa/L. BMI, body mass index; SD, standard deviation; sIgE, allergen-specific immunoglobulin E; SPT, skin prick test.

TABLE 2
Mean TNSS Reduction from Placebo

| | Day 29 | Day 57 | Day 85 |
|---|---|---|---|
| | Reduction in Mean | Reduction in | Reduction in |
| | TNSS/% Reduction | Mean TNSS | Mean TNSS |
| REGN5715 | −1.51 (p = 0.0023) | −0.71 (p = 0.2128) | −0.77 (p = 0.2128) |
| | 22.5% | | |
| REGN5713 + | −2.65 (p < 0.0001) | −2.4 (p < 0.0001) | −1.66 (p = 0.0081) |
| REGN5715 | 39.5% | | |
| REGN5713 + | −2.31 (p < 0.0001) | −1.83 (p = 0.0017) | −1.54 (p = 0.0148) |
| REGN5715 + | 34.4% | | |
| REGN5714 | | | |

### CONCLUSION

A single dose of a combination of Bet v 1 antibodies provided robust and durable reduction in birch pollen induced allergic nasal symptoms for up to 3 months, which is expected to provide coverage for the duration of an entire natural birch season. Surprisingly, a single dose of the REGN5713/REGN5715 two-antibody combination was at least as efficacious as the REGN5713/REGN5714/REGN5715 three-antibody combination.

The finding that REGN5713/REGN5715 was as efficacious as the REGN5713/REGN5714/REGN5715 three-antibody combination is surprising in view of previously reported in vitro data comparing single anti-Bet v 1 antibodies and combinations of antibodies (Atanasio et al., J Allergy Clin Immunol 2022, 149:200-211). A humanized Passive Cutaneous Anaphylaxis mouse model was used that has been shown to translate to efficacy in individuals with allergy (Orengo et al., Nat Commun 2018, 9:1421). The mice were sensitized with human plasma containing polyclonal birch specific-IgE from 5 human birch-allergic donors, then challenged systemically with natural Bet v 1. REGN5713-5714-5715 achieved 90% blockade of mast cell degranulation in ⅘ donors evaluated, while REGN5713-5715 achieved the same magnitude of blockade in only ⅕ donors evaluated. As compared with the single mAb or double mAb combination, these in vitro data suggested that greater blockade of Bet v 1-induced allergy would be achieved with a REGN5713-5714-5715 triple antibody cocktail.

Additionally, the comparable efficacy of REGN5713/REGN5715 to a REGN5713/REGN5714/REGN5715 three-antibody combination is surprising in view of what was known about the interaction of these antibodies with the Bet v 1 antigen. Atanasio discloses the cryo-EM structure of natural Bet v 1 bound to REGN5713, REGN5714, and REGN5715 Fabs, and shows that simultaneous binding of REGN5713 and REGN5715 to Bet v 1 still leaves more than half of the surface area of Bet v 1 exposed. REGN5714 binds in part of this exposed region of Bet v 1, thus leaving less exposed surface area of Bet v 1 when a REGN5713-5714-5715 triple antibody cocktail is used, and accordingly supported the hypothesis that a REGN5713-5714-5715 triple antibody cocktail would provide greater treatment effect than a two-antibody cocktail due to broader epitope coverage.

### Example 2: Clinical Trial to Assess the Efficacy and Safety of REGN5713 and REGN5715 in Reducing Signs and Symptoms of Allergic Conjunctivitis in Participants with Birch Pollen Allery

### Study Design and Objectives

This study is a randomized, double-masked, placebo-controlled study in birch allergic participants to demonstrate the efficacy of REGN5713-5715 on the reduction of allergic conjunctivitis signs and symptoms during exposure to birch allergen during conjunctival allergen challenges (CACs). The study will assess whether a single dose of REGN5713-5715 (600 mg) reduces ocular itch (symptom) and conjunctival redness (sign) during a birch CAC, relative to placebo, in addition to evaluating the efficacy in reduction of other allergic signs and symptoms (such as tearing, chemosis, eyelid swelling, TOSS, and nasal symptoms). Additionally, the efficacy of REGN5713-5715 (600 mg) versus placebo in the reduction of skin test reactivity to birch will be assessed. Similarly, efficacy in the reduction of oak induced allergic conjunctivitis signs and symptoms, as well as skin test reactivity, will be explored.

The primary endpoint for the study is ocular itch score at 3, 5, and 7 minutes post-CAC at day 8. Ocular itch score is reported by the participant using the Ora Calibra® Conjunctival Allergen Challenge Ocular Itching Scale (0 to 4 with 0.5 unit increments).

The key secondary endpoints for the study are: (i) conjunctival redness score at 7, 15, and 20 minutes post-CAC at day 8, assessed by the investigator with a slit lamp using the Ora Calibra® Ocular Hyperemia Scale (for conjunctival, ciliary, and episcleral vessel beds using a slit lamp: 0 to 4 with 0.5 unit increments); and (ii) percent change from baseline in birch titrated skin prick test (tSPT) (AUC of the mean wheal diameter) at day 8.

Other secondary endpoints for the study include: (i) tearing score at 7, 15, and 20 minutes post-CAC at day 8, reported by the participant using the Ora Calibra® Conjunctival Allergen Challenge Tearing Scale (0 to 4 with 1 unit increments); (ii) TOSS post-CAC at day 8; (iii) total redness score at 7, 15, and 20 minutes post-CAC at day 8; (iv) ciliary redness score at 7, 15, and 20 minutes post-CAC at day 8, assessed by the investigator with a slit lamp using the Ora Calibra® Ocular Hyperemia Scale (for conjunctival, ciliary, and episcleral vessel beds using a slit lamp: 0 to 4 with 0.5 unit increments); (v) episcleral redness score at 7, 15, and 20 minutes post-CAC at day 8, assessed by the investigator with a slit lamp using the Ora Calibra® Ocular Hyperemia Scale (for conjunctival, ciliary, and episcleral vessel beds using a slit lamp: 0 to 4 with 0.5 unit increments); (vi) change from baseline in birch tSPT (AUC of the mean wheal diameters) at day 8; (vii) change and percent change from baseline in birch tSPT (AUC of the mean wheal diameters) at days 22 and 11; (viii) change and percent change from baseline in oak tSPT (AUC of the mean wheal diameters) at day 8; (ix) incidence of TEAEs, serious TEAEs, and AESIs; (x) total REGN7513 and REGN5715 concentrations in serum over time; and (xi) incidence and titers of ADAs to REGN7513 and REGN5715.

Approximately 50 adult participants, aged 18 years and older, with a history of moderate to severe birch pollen allergy for at least 2 prior birch seasons will be enrolled in this study.

### Key Inclusion Criteria

A participant must meet the following criteria to be eligible for inclusion in the study:
Adult participant aged 18 years or older at the time of screening visit 1
Documented or participant-reported history of moderate to severe birch pollen allergy for at least 2 years with bothersome ocular symptoms during the birch season
Positive SPT to birch allergen extract (mean wheal diameter at least 5 mm greater than the negative control) at screening visit 1
Positive slgE tests for birch and Bet v 1 (both ≥0.7 kUa/L) at screening visit 1.
Must be able to complete birch screening CACs and meet the following criteria as defined below to confirm moderate to severe birch induced allergic conjunctivitis: (a) Bilateral positive CAC reactions* within approximately 10 minutes of birch allergen instillation during the birch screening titration CAC, AND (b) Bilateral positive CAC reactions in at least 2 out of 3 post-CAC time points, following instillation of the birch eliciting allergen dose (see Section 5.1), assessed during the birch confirmatory CAC. *Bilateral positive CAC reactions are defined as ocular itching ≥ 2/4 and conjunctival redness ≥ 2/4 in both eyes.
Have a calculated best-corrected visual acuity of 0.7 logarithm of the minimum angle of resolution (Log MAR) (equivalent to 6/30 Snellen equivalence, or 20/100 vision) or better in each eye as measured using an ETDRS chart
Willing and able to comply with clinic visits and study-related procedures
Provide informed consent signed by study participant or legally acceptable representative
Able to understand and complete study-related questionnaires

### Key Exclusion Criteria

A participant who meets any of the following criteria will be excluded from the study:
Participation in a prior clinical study and received either REGN5713-5714-5715, REGN5713-5715, or REGN5715 antibodies (receipt of placebo in a previous study is allowed)
Inability to complete or termination of the screening or confirmatory CACs due to a safety concern (e.g., anaphylaxis) per PI judgment
Significant and/or severe allergies, ocular or nasal diseases or systemic diseases, causing symptoms (e.g., ocular itching, ocular redness, etc.) that are expected to coincide or potentially interfere with the study CAC assessments, as assessed by the investigator (e.g., clinically active allergic conjunctivitis at screening visit 1 or prior to start of CAC at visit 2, significant atopic keratoconjunctivitis, vernal keratoconjunctivitis, perennial symptomatic allergy [e.g., symptomatic perennial allergic conjunctivitis due to house dust mite allergy], animal allergy with expected exposure [e.g., cat allergy and lives with a cat], recurrent sinusitis, nasal polyps, etc.)
Persistent chronic or recurring acute infection requiring treatment with systemic antibiotics, antivirals, or antifungals, or any untreated respiratory infections within 4 weeks prior to screening visit 1. Participants may be re-evaluated for eligibility after resolution of symptoms and specified duration (participants with isolated oral herpes simplex virus requiring short term use of antiviral therapy may be permitted, per PI discretion)
The presence of an active ocular infection (bacterial, viral, or fungal) or diagnosis by a physician within 30 days prior to screening visit 1 (participants may be re-evaluated for eligibility after resolution of symptoms and specified duration). Participants with a history of an ocular herpetic infection will be excluded.
Current or prior history of glaucoma or ocular hypertension
Presence of ocular conditions such as blepharitis, active rosacea affecting the ocular adnexa, meibomian gland dysfunction, follicular conjunctivitis, intraocular inflammation, preauricular lymphadenopathy, any ocular condition associated with acute or chronic vision loss, or any other ophthalmic disease or abnormality that may interfere with study assessments, affect the study outcomes or participant safety, per PI judgment
Any ocular surgical intervention and/or a history of refractive surgery (including ocular laser procedures) within 6 months of screening visit 1
Confirmed diagnosis of dry eye (signs and symptoms)
Use of prohibited medications during the specified periods
Abnormal lung function as judged by the investigator with FEV1<70% of predicted at screening visit 1
Current (within 12 months prior to screening visit 1) moderate to severe asthma or uncontrolled asthma, or on GINA steps 4 to 5 (Global Initiative for Asthma, 2024) (e.g., controlled asthmatics requiring intermittent use of a short acting bronchodilator±ICS or intermittent ICS-LABA or low dose ICS/ICS-LABA may be considered acceptable; per PI judgment)
Documented or reported clinical history of tree pollen induced severe asthma exacerbations during 2 prior tree pollen seasons requiring the use of systemic steroids and/or hospitalization or emergency room/urgent care visit, per PI judgment
A clinical history of asthma with treatment of asthma requiring systemic (oral or parenteral) corticosteroid treatment more than twice within prior 12 months or once within 3 months prior to screening visit 1 or has been hospitalized or has attended the Emergency Room/Urgent Care facility for asthma in the 12 months prior to screening.
History of life-threatening asthma, defined as an asthma episode that required intubation and/or was associated with hypercapnia, respiratory arrest, and/or hypoxic seizures.
Active lung disease other than asthma
History birch or other tree AIT (e.g., SCIT, SLIT, or any other route) in the 3 years prior to screening visit 1.
Ongoing AIT with any allergen other than birch or other trees at screening visit 1. Participants may be re-evaluated for eligibility after discontinuation of AIT or may be eligible if discontinued at screening visit 1.
Use of other biological therapy (including but not limited to biologics with anti-IgE, anti-IL5, anti-IL4Ra, anti-IL13, or anti-TSLP) that interferes with type 2 disease within 6 months prior to screening visit 1
History of, current, or recent (<12 months before screening visit 1) severe, unstable, or uncontrolled autoimmune, neurologic, cardiovascular, hematologic, hepatic, renal, psychiatric, respiratory, gastrointestinal, metabolic and/or immunologic disease or evidence of other diseases based on a review of medical history and/or physical examination (e.g., vital signs, electrocardiogram, clinical laboratory test results) that, in the opinion of the investigator, would preclude safe subject participation in the study or may confound the study results

### Study Drug

For this trial, participants will receive subcutaneous administration of either REGN5713-5715 at a dose of 600 mg (300 mg per mAb), or matching placebo that replaces active drug. Study drug will be provided as follows:
REGN5713, lyophilized, 265 mg in 20 mL vial
REGN5715, lyophilized, 265 mg in 20 mL vial
Placebo, lyophilized, 20 mL vial

For REGN5713-5715, the 1:1 mixture of reconstituted drug product results in a solution of 150 mg/mL REGN5713-5715 (75 mg/mL REGN5713; 75 mg/mL REGN5715). Participants randomized to receive 600 mg REGN5713-5715 will receive 2 injections of 2 mL, each administered as SC injections of REGN5713-5715 (both antibodies co-administered in each injection).

Treatment for acute and/or severe reactions is allowed during the study. Allergen exposure can induce immediate or late allergic reactions, such as allergic conjunctivitis, allergic rhinitis, asthma symptoms, or rarely anaphylaxis, in sensitized participants, which will be treated appropriately at the discretion of the investigator. For treatment of severe or clinically concerning reactions (as judged by the investigator), rescue treatments may be used during the CAC sessions that may include but are not limited to topical/systemic antihistamines, epinephrine, topical/systemic corticosteroids, and/or SABA (e.g., albuterol/salbutamol/terbutaline), per PI judgment. With the exception of severe or clinically concerning reactions (as judged by the investigator), anti-allergy treatments (e.g., antihistamines, vasoconstrictors), as needed, may be used after study visit endpoint data collection is obtained (e.g., after CAC data are collected, after the skin wheals are measured after application of allergen during SPT).

### Efficacy Procedures

Procedures for assessing efficacy are described below:

Ocular Itch Score: Ocular itch score is reported by the participant using the Ora Calibra® Conjunctival Allergen Challenge Ocular Itching Scale (0 to 4 with 0.5 unit increments; site-administered) at specified timepoints. The titration CACs (birch screening titration CAC and oak screening titration CAC) will assess these scores prior to the CAC (pre-CAC score) and within approximately 10 minutes of allergen instillation until bilateral positive CAC reactions are elicited. The confirmatory CAC at screening and the efficacy CAC will assess these scores prior to the CAC (pre-CAC score) and at approximately 3, 5, and 7 minutes post CAC allergen administration.

Ocular Redness Score: Conjunctival redness, ciliary redness, and episcleral redness scores each are assessed by the investigator with a slit lamp using the Ora Calibra® Ocular Hyperemia Scale (for conjunctival, ciliary, and episcleral vessel beds using a slit lamp: 0 to 4 with 0.5 unit increments) at specified timepoints. The titration CACs (birch screening titration CAC and oak screening titration CAC) will assess conjunctival redness, ciliary redness, and episcleral redness scores prior to the CAC (pre-CAC score) and within approximately 10 minutes of allergen instillation until bilateral positive CAC reactions are elicited. The confirmatory CAC at screening and efficacy CAC will assess conjunctival redness, ciliary redness, and episcleral redness scores prior to the CAC (pre-CAC score) and at approximately 7, 15, and 20 minutes post CAC allergen administration.

Total Redness Score: The total redness score is calculated for CAC as a sum of the bilateral averages for conjunctival redness score+ciliary redness score+episcleral redness score) (range: 0 to 12).

Tearing Score: The tearing score is reported by the participant, using the Ora Calibra® Conjunctival Allergen Challenge Tearing Scale (0 to 4 with 1 unit increments; site-administered), at specified timepoints. The titration CACs (birch screening titration CAC and oak screening titration CAC) will assess tearing prior to the CAC (pre-CAC score) and if an eliciting allergen dose is identified then tearing will be assessed within approximately 10 minutes of instilling the eliciting allergen dose. The confirmatory CAC at screening and efficacy CAC will assess these scores prior to the CAC (pre-CAC score) and at approximately 7, 15, and 20 minutes post-CAC allergen administration.

Total Ocular Symptom Score (TOSS): TOSS is a summed score of ocular itch score (graded 0 to 4, described above), conjunctival redness score (graded 0 to 4, described above), and tearing score (graded 0 to 4, described above) for a total range from 0 to 12. TOSS is a sum of the averaged ocular itch score pre-CAC and at approximately 3, 5, and 7 minutes post-CAC, averaged tearing score pre CAC and at approximately 7, 15, and 20 minutes post CAC, and averaged bilateral conjunctival redness scores pre CAC and at approximately 7, 15, and 20 minutes post-CAC allergen administration. Pre-CAC and post-CAC TOSS scores will be calculated during the confirmatory CAC at screening and the efficacy CAC.

Chemosis Score: Chemosis score is assessed by the investigator using the Ora Calibra® Chemosis Scale using slit lamp (0 to 4 with 0.5 unit increments) at specified timepoints. The titration CACs (birch screening titration CAC and oak screening titration CAC) will assess chemosis prior to the CAC (pre-CAC score) and if an eliciting allergen dose is identified then chemosis will be assessed within approximately 10 minutes of instilling the eliciting allergen dose. The confirmatory CAC at screening and the efficacy CAC will assess these scores prior to the CAC (pre-CAC score) and at approximately 7, 15, and 20 minutes post CAC allergen administration.

Eyelid Swelling Score: Eyelid swelling score is reported by the participant, using the Ora Calibra® Conjunctival Allergen Challenge Eyelid Swelling Scale (0 to 3 with 1 unit increments; site administered) at specified timepoints. The titration CACs (birch screening titration CAC and oak screening titration CAC) will assess eyelid swelling score prior to the CAC (pre-CAC score) and if an eliciting allergen dose is identified then eyelid swelling score will be assessed within approximately 10 minutes of instilling the eliciting allergen dose. The confirmatory CAC at screening and the efficacy CAC will assess these scores prior to the CAC (pre-CAC score) and at approximately 7, 15, and 20 minutes post CAC allergen administration.

Total Nasal Symptom Score (TNSS) and Components: TNSS is reported by the participants at specified timepoints. The TNSS ranges from 0 to 12 and is based on assessment of 4 nasal symptoms graded on a Likert scale ranging from 0 (none) to 3 (severe) for nasal congestion, nasal itching, rhinorrhea, and sneezing. The titration CACs (birch screening titration CAC and oak screening titration CAC) will assess TNSS prior to the CAC (pre-CAC score) and if an eliciting allergen dose is identified then TNSS will be assessed within approximately 10 minutes of instilling the eliciting allergen dose. The confirmatory CAC at screening and the efficacy CAC will assess these scores prior to the CAC (pre-CAC score) and at approximately 20 minutes post CAC allergen administration.

Ear/Palate Pruritus Score: Ear/palate pruritus score ranges from 0 to 3 (1 unit increments; 0 [none] and 3 [severe]) and is reported by the participant at specified timepoints. The titration CACs (birch screening titration CAC and oak screening titration CAC) will assess ear/palate pruritus score prior to the CAC (pre-CAC score) and if an eliciting allergen dose is identified then ear/palate pruritus score will be assessed within approximately 10 minutes of instilling the eliciting allergen dose. The confirmatory CAC at screening and the efficacy CAC will assess these scores prior to the CAC (pre-CAC score) and at approximately 20 minutes post CAC allergen administration.

Informal Sequence Listing

| SEQ ID | | |
|---|---|---|
| NO | Sequence | Description |
| 1 | QVQLQESGPGLVKPSETLSLTCSVSGGSITNYFWTWIRQSPGKGLEWIGYIYYSGGTNYNP | REGN5713 heavy |
| | SLKSRVTISIDTSKNQFSLNMNSVTAADTAVYYCAGSYYYGVDVWGQGTTVTVSS | chain variable region |
| 2 | GGSITNYF | REGN5713 HCDR1 |
| 3 | IYYSGGT | REGN5713 HCDR2 |
| 4 | AGSYYYGVDV | REGN5713 HCDR3 |
| 5 | EIVLTQSPATLSLSPGERATLSCRASQSIKSFLAWYRQKPGQAPRLLIYDASNRPTGIPARFS | REGN5713 light chain |
| | GSGSGTDFTLTINSLESEDFAVYFCQQRNNWPFTFGPGTKVDIK | variable region |
| 6 | QSIKSF | REGN5713 LCDR1 |
| 7 | DAS | REGN5713 LCDR2 |
| 8 | QQRNNWPFT | REGN5713 LCDR3 |
| 9 | QVQLQESGPGLVKPSETLSLTCSVSGGSITNYFWTWIRQSPGKGLEWIGYIYYSGGTNYNP | REGN5713 heavy |
| | SLKSRVTISIDTSKNQFSLNMNSVTAADTAVYYCAGSYYYGVDVWGQGTTVTVSSASTKG | chain |
| | PSVFPLAPCSRSTSESTAALGCLVKDYFPEPVTVSWNSGALTSGVHTFPAVLQSSGLYSLSS | |
| | VVTVPSSSLGTKTYTCNVDHKPSNTKVDKRVESKYGPPCPPCPAPEFLGGPSVFLFPPKPKD | |
| | TLMISRTPEVTCVVVDVSQEDPEVQFNWYVDGVEVHNAKTKPREEQFNSTYRVVSVLTVL | |
| | HQDWLNGKEYKCKVSNKGLPSSIEKTISKAKGQPREPQVYTLPPSQEEMTKNQVSLTCLVK | |
| | GFYPSDIAVEWESNGQPENNYKTTPPVLDSDGSFFLYSRLTVDKSRWQEGNVFSCSVMH | |
| | EALHNHYTQKSLSLSLGK | |
| 10 | EIVLTQSPATLSLSPGERATLSCRASQSIKSFLAWYRQKPGQAPRLLIYDASNRPTGIPARFS | REGN5713 light chain |
| | GSGSGTDFTLTINSLESEDFAVYFCQQRNNWPFTFGPGTKVDIKRTVAAPSVFIFPPSDEQL | |
| | KSGTASVVCLLNNFYPREAKVQWKVDNALQSGNSQESVTEQDSKDSTYSLSSTLTLSKADY | |
| | EKHKVYACEVTHQGLSSPVTKSFNRGEC | |
| 11 | EVQLVESGGDLVQPGGSLRLSCAASGFTFSSYEMNWVRQAPGKGLEWVSFISDSSSNIYY | REGN5714 heavy |
| | ADSVKGRFTISRDNAKKSLYLQMTSLRAEDTAVYYCAREAIGSTSFDNWGQGTLVTVSS | chain variable region |
| 12 | GFTFSSYE | REGN5714 HCDR1 |
| 13 | ISDSSSNI | REGN5714 HCDR2 |
| 14 | AREAIGSTSFDN | REGN5714 HCDR3 |
| 15 | EIVMTQSPATLSVSPGERATLSCRASQSVSSSLAWYQQKPGQAPRRLIYSASTRATGIPARF | REGN5714 light chain |
| | SGSGSGTEFTLTISSLQSEDFAIYYCHQYNNWPLTFGGGTKVEIK | variable region |
| 16 | QSVSSS | REGN5714 LCDR1 |
| 17 | SAS | REGN5714 LCDR2 |
| 18 | HQYNNWPLT | REGN5714 LCDR3 |
| 19 | EVQLVESGGDLVQPGGSLRLSCAASGFTFSSYEMNWVRQAPGKGLEWVSFISDSSSNIYY | REGN5714 heavy |
| | ADSVKGRFTISRDNAKKSLYLQMTSLRAEDTAVYYCAREAIGSTSFDNWGQGTLVTVSSAS | chain |
| | TKGPSVFPLAPCSRSTSESTAALGCLVKDYFPEPVTVSWNSGALTSGVHTFPAVLQSSGLYS | |
| | LSSVVTVPSSSLGTKTYTCNVDHKPSNTKVDKRVESKYGPPCPPCPAPEFLGGPSVFLFPPK | |
| | PKDTLMISRTPEVTCVVVDVSQEDPEVQFNWYVDGVEVHNAKTKPREEQFNSTYRVVSV | |
| | LTVLHQDWLNGKEYKCKVSNKGLPSSIEKTISKAKGQPREPQVYTLPPSQEEMTKNQVSLT | |
| | CLVKGFYPSDIAVEWESNGQPENNYKTTPPVLDSDGSFFLYSRLTVDKSRWQEGNVFSCS | |
| | VMHEALHNHYTQKSLSLSLGK | |
| 20 | EIVMTQSPATLSVSPGERATLSCRASQSVSSSLAWYQQKPGQAPRRLIYSASTRATGIPARF | REGN5714 light chain |
| | SGSGSGTEFTLTISSLQSEDFAIYYCHQYNNWPLTFGGGTKVEIKRTVAAPSVFIFPPSDEQL | |
| | KSGTASVVCLLNNFYPREAKVQWKVDNALQSGNSQESVTEQDSKDSTYSLSSTLTLSKADY | |
| | EKHKVYACEVTHQGLSSPVTKSFNRGEC | |
| 21 | QVQLVQSGAEVKKPGASVKVSCKASGYTFISYNIFWVRQATGQGLDWMGWMNPFRNN | REGN5715 heavy |
| | AGYAQKFQGRVTVTWDTSISTAYMELSSLSSEDTAIYYCAREHGSSWGFFDYWGQGTLVT | chain variable region |
| | VSS | |
| 22 | GYTFISYN | REGN5715 HCDR1 |
| 23 | MNPFRNNA | REGN5715 HCDR2 |
| 24 | AREHGSSWGFFDY | REGN5715 HCDR3 |
| 25 | EIVLTQSPGTLSLSPGERATLSCRASQSVSSSYLAWYQQKPGQAPRLLIYGASSRATGIPDRF | REGN5715 light chain |
| | SGSGSGTDFTLTISRLEPEDFAVYYCQQYGSSPWTFGQGTKVEIK | variable region |
| 26 | QSVSSSY | REGN5715 LCDR1 |
| 27 | GAS | REGN5715 LCDR2 |
| 28 | QQYGSSPWT | REGN5715 LCDR3 |
| 29 | QVQLVQSGAEVKKPGASVKVSCKASGYTFISYNIFWVRQATGQGLDWMGWMNPFRNN | REGN5715 heavy |
| | AGYAQKFQGRVTVTWDTSISTAYMELSSLSSEDTAIYYCAREHGSSWGFFDYWGQGTLVT | chain |
| | VSSASTKGPSVFPLAPCSRSTSESTAALGCLVKDYFPEPVTVSWNSGALTSGVHTFPAVLQS | |
| | SGLYSLSSVVTVPSSSLGTKTYTCNVDHKPSNTKVDKRVESKYGPPCPPCPAPEFLGGPSVF | |
| | LFPPKPKDTLMISRTPEVTCVVVDVSQEDPEVQFNWYVDGVEVHNAKTKPREEQFNSTYR | |
| | VVSVLTVLHQDWLNGKEYKCKVSNKGLPSSIEKTISKAKGQPREPQVYTLPPSQEEMTKN | |
| | QVSLTCLVKGFYPSDIAVEWESNGQPENNYKTTPPVLDSDGSFFLYSRLTVDKSRWQEGN | |
| | VFSCSVMHEALHNHYTQKSLSLSLGK | |
| 30 | EIVLTQSPGTLSLSPGERATLSCRASQSVSSSYLAWYQQKPGQAPRLLIYGASSRATGIPDRF | REGN5715 light chain |
| | SGSGSGTDFTLTISRLEPEDFAVYYCQQYGSSPWTFGQGTKVEIKRTVAAPSVFIFPPSDEQ | |
| | LKSGTASVVCLLNNFYPREAKVQWKVDNALQSGNSQESVTEQDSKDSTYSLSSTLTLSKAD | |
| | YEKHKVYACEVTHQGLSSPVTKSFNRGEC | |
| 31 | MGVFNYETETTSVIPAARLFKAFILDGDNLFPKVAPQAISSVENIEGNGGPGTIKKISFPEGL | Bet v 1 amino acid |
| | PFKYVKDRVDEVDHTNFKYNYSVIEGGPIGDTLEKISNEIKIVATPDGGSILKISNKYHTKGD | sequence from |
| | HEVKAEQVKASKEMGETLLRAVESYLLAHSDAYN | CAB02159 |
| 32 | MGVFNYETETTSVIPAARLFKAFILDGDNLFPKVAPQAISSVENIEGNGGPGTIKKISFPEGF | Bet v 1 amino acid |
| | PFKYVKDRVDEVDHTNFKYNYSVIEGGPIGDTLEKISNEIKIVATPDGGSILKISNKYHTKGD | sequence from |
| | HEVKAEQVKASKEMGETLLRAVESYLLAHSDAYN | Uniprot P15494 |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims. The disclosures of all patents and non-patent literature cited herein are expressly incorporated in their entirety by reference.

1. A method of treating one or more symptoms of birch allergy in a subject, the method comprising administering to the subject:
a first anti-Bet v 1 antibody or antigen-binding fragment thereof, wherein the first anti-Bet v 1 antibody or antigen-binding fragment thereof comprises a heavy chain complementarity determining region (HCDR) 1 comprising the amino acid sequence of SEQ ID NO:2, an HCDR2 comprising the amino acid sequence of SEQ ID NO:3, an HCDR3 comprising the amino acid sequence of SEQ ID NO:4, a light chain complementarity determining region (LCDR) 1 comprising the amino acid sequence of SEQ ID NO:6, an LCDR2 comprising the amino acid sequence of SEQ ID NO:7, and an LCDR3 comprising the amino acid sequence of SEQ ID NO: 8; and
a second anti-Bet v 1 antibody or antigen-binding fragment thereof, wherein the second anti-Bet v 1 antibody or antigen-binding fragment thereof comprises an HCDR1 comprising the amino acid sequence of SEQ ID NO:22, an HCDR2 comprising the amino acid sequence of SEQ ID NO: 23, an HCDR3 comprising the amino acid sequence of SEQ ID NO:24, an LCDR1 comprising the amino acid sequence of SEQ ID NO:26, an LCDR2 comprising the amino acid sequence of SEQ ID NO:27, and an LCDR3 comprising the amino acid sequence of SEQ ID NO: 28;
wherein the method does not comprise administering a third anti-Bet v 1 antibody to the subject.

2. The method of claim 1, wherein the method comprises administering to the subject a single dose of each of the first anti-Bet v 1 antibody or antigen-binding fragment thereof and the second anti-Bet v 1 antibody or antigen-binding fragment thereof prior to the start of birch pollen season.

3. The method of claim 1, wherein each of the first anti-Bet v 1 antibody or antigen-binding fragment thereof and the second anti-Bet v 1 antibody or antigen-binding fragment thereof is administered at a dose of about 150 mg to about 300 mg.

4. The method of claim 1, wherein the first anti-Bet v 1 antibody or antigen-binding fragment thereof and the second anti-Bet v 1 antibody or antigen-binding fragment thereof are formulated separately.

5. The method of claim 1, wherein the first anti-Bet v 1 antibody or antigen-binding fragment thereof and the second anti-Bet v 1 antibody or antigen-binding fragment thereof are co-formulated in a pharmaceutical composition.

6. The method of claim 5, wherein the pharmaceutical composition consists essentially of the first anti-Bet v 1 antibody or antigen-binding fragment thereof and the second anti-Bet v 1 antibody or antigen-binding fragment thereof.

7. The method of claim 5, wherein the pharmaceutical composition comprises the first anti-Bet v 1 antibody or antigen-binding fragment thereof and the second anti-Bet v 1 antibody or antigen-binding fragment thereof at a total dose of about 300 mg.

8. The method of claim 7, wherein the pharmaceutical composition comprises each of the first anti-Bet v 1 antibody or antigen-binding fragment thereof and the second anti-Bet v 1 antibody or antigen-binding fragment thereof at a dose of about 150 mg.

9. The method of claim 5, wherein the pharmaceutical composition comprises the first anti-Bet v 1 antibody or antigen-binding fragment thereof and the second anti-Bet v 1 antibody or antigen-binding fragment thereof at a total dose of about 600 mg.

10. The method of claim 9, wherein the pharmaceutical composition comprises each of the first anti-Bet v 1 antibody or antigen-binding fragment thereof and the second anti-Bet v 1 antibody or antigen-binding fragment thereof at a dose of about 300 mg.

11. The method of claim 1, wherein the first anti-Bet v 1 antibody or antigen-binding fragment thereof and the second anti-Bet v 1 antibody or antigen-binding fragment thereof are administered subcutaneously.

12. The method of claim 1, wherein the first anti-Bet v 1 antibody or antigen-binding fragment thereof and the second anti-Bet v 1 antibody or antigen-binding fragment thereof are administered intravenously.

13. The method of claim 1, wherein the first anti-Bet v 1 antibody or antigen-binding fragment thereof comprises a heavy chain variable region (HCVR) comprising the amino acid sequence of SEQ ID NO:1 and a light chain variable region (LCVR) comprising the amino acid sequence of SEQ ID NO:5.

14. The method of claim 1, wherein the first anti-Bet v 1 antibody or antigen-binding fragment thereof comprises a heavy chain comprising the amino acid sequence of SEQ ID NO:9 and a light chain comprising the amino acid sequence of SEQ ID NO:10.

15. The method of claim 1, wherein the first anti-Bet v 1 antibody is bremzalerbart.

16. The method of claim 1, wherein the second anti-Bet v 1 antibody or antigen-binding fragment thereof comprises an HCVR comprising the amino acid sequence of SEQ ID NO:21 and an LCVR comprising the amino acid sequence of SEQ ID NO:25.

17. The method of claim 1, wherein the second anti-Bet v 1 antibody or antigen-binding fragment thereof comprises a heavy chain comprising the amino acid sequence of SEQ ID NO:29 and a light chain comprising the amino acid sequence of SEQ ID NO:30.

18. The method of claim 1, wherein the second anti-Bet v 1 antibody is atisnolerbart.

19. The method of claim 1, wherein treatment with the first anti-Bet v 1 antibody or antigen-binding fragment thereof and the second anti-Bet v 1 antibody or antigen-binding fragment thereof:
reduces a subject's Total Nasal Symptom Score (TNSS);
reduces a subject's Total Ocular Symptom Score (TOSS);
reduces a subject's Total Symptom Score (TSS);
reduces a subject's Daily Medication Score (DMS);
reduces a subject's Combined Symptom and Medication Score (CSMS);
reduces a subject's birch skin prick test (SPT) mean wheal diameter; and/or
increases a subject's number of well days in which rescue medication is not utilized and the subject's TSS is ≤2 of 18.

20. The method of claim 19, wherein treatment with the first anti-Bet v 1 antibody or antigen-binding fragment thereof and the second anti-Bet v 1 antibody or antigen-binding fragment thereof reduces a subject's TNSS, TOSS, TSS, DMS, and/or CSMS for the duration of birch pollen season.

21. The method of claim 1, wherein treatment with the first anti-Bet v 1 antibody or antigen-binding fragment thereof and the second anti-Bet v 1 antibody or antigen-binding fragment thereof reduces allergic rhinitis symptoms in the subject.

22. The method of claim 21, wherein treatment with the first anti-Bet v 1 antibody or antigen-binding fragment thereof and the second anti-Bet v 1 antibody or antigen-binding fragment thereof reduces a subject's mean Total Nasal Symptom Score (TNSS) after nasal allergen challenge (NAC) by at least about 30%, relative to TNSS after NAC in the absence of treatment.

23. The method of claim 22, wherein administration of a single dose of the first anti-Bet v 1 antibody or antigen-binding fragment thereof and the second anti-Bet v 1 antibody or antigen-binding fragment thereof reduces a subject's mean TNSS after NAC by at least about 30% for at least about 29 days after the pharmaceutical composition is administered relative to TNSS after NAC in the absence of treatment.

24. The method of claim 1, wherein treatment with the first anti-Bet v 1 antibody or antigen-binding fragment thereof and the second anti-Bet v 1 antibody or antigen-binding fragment thereof reduces allergic conjunctivitis symptoms in the subject.

25. The method of claim 1, wherein treatment with the first anti-Bet v 1 antibody or antigen-binding fragment thereof and the second anti-Bet v 1 antibody or antigen-binding fragment thereof reduces birch sensitization in the subject as measured by a skin prick test (SPT) with a birch allergen extract relative to birch sensitization in the absence of treatment with the pharmaceutical composition.

26. The method of claim 1, wherein treatment with the first anti-Bet v 1 antibody or antigen-binding fragment thereof and the second anti-Bet v 1 antibody or antigen-binding fragment thereof reduces ocular itch in the subject.

27. The method of claim 1, wherein treatment with the first anti-Bet v 1 antibody or antigen-binding fragment thereof and the second anti-Bet v 1 antibody or antigen-binding fragment thereof reduces conjunctival redness in the subject.

28. The method of claim 1, wherein the subject to be treated has a baseline serum allergen-specific IgE level ≥0.7 kUa/L for birch tree pollen and/or Bet v 1 allergen.

29. The method of claim 1, wherein the subject to be treated has a baseline positive SPT with a birch allergen extract.

30. A method of treating one or more signs or symptoms associated with allergic conjunctivitis in a subject having birch tree pollen allergy, the method comprising administering to the subject:
a first anti-Bet v 1 antibody, wherein the first anti-Bet v 1 antibody comprises a heavy chain variable region (HCVR) comprising the amino acid sequence of SEQ ID NO:1 and a light chain variable region (LCVR) comprising the amino acid sequence of SEQ ID NO:5; and
a second anti-Bet v 1 antibody or antigen-binding fragment thereof, wherein the second anti-Bet v 1 antibody comprises an HCVR comprising the amino acid sequence of SEQ ID NO: 21 and an LCVR comprising the amino acid sequence of SEQ ID NO:25;
wherein the method does not comprise administering a third anti-Bet v 1 antibody to the subject.

31. The method of claim 30, wherein the subject is an adult.

32. The method of claim 30, wherein the subject:
(i) has a baseline serum allergen-specific IgE level ≥0.7 kUa/L for birch tree pollen and/or Bet v 1 allergen;
(ii) has a baseline birch skin prick test (SPT) mean wheal diameter ≥5 mm;
(iii) has a baseline ocular itch score ≥2 in both eyes after a conjunctival allergen challenge (CAC), as measured using the Ora Calibra® Conjunctival Allergen Challenge Ocular Itching Scale; and/or
(iv) has a baseline conjunctival redness score ≥2 in both eyes after a CAC, as measured using the Ora Calibra® Ocular Hyperemia Scale.

33. The method of claim 30, wherein the method comprises administering to the subject a single dose of each of the first anti-Bet v 1 antibody and the second anti-Bet v 1 antibody prior to the start of birch pollen season.

34. The method of claim 30, wherein each of the first anti-Bet v 1 antibody and the second anti-Bet v 1 antibody is administered at a dose of about 300 mg.

35. The method of claim 30, wherein the first anti-Bet v 1 antibody comprises a heavy chain comprising the amino acid sequence of SEQ ID NO:9 and a light chain comprising the amino acid sequence of SEQ ID NO:10.

36. The method of claim 30, wherein the first anti-Bet v 1 antibody is bremzalerbart.

37. The method of claim 30, wherein the second anti-Bet v 1 antibody comprises a heavy chain comprising the amino acid sequence of SEQ ID NO:29 and a light chain comprising the amino acid sequence of SEQ ID NO:30.

38. The method of claim 30, wherein the second anti-Bet v 1 antibody is atisnolerbart.

39. The method of claim 30, wherein the first anti-Bet v 1 antibody and the second anti-Bet v 1 antibody are administered subcutaneously.

40. The method of claim 30, wherein the first anti-Bet v 1 antibody and the second anti-Bet v 1 antibody are co-formulated in a pharmaceutical composition.

41. The method of claim 40, wherein the pharmaceutical composition consists essentially of the first anti-Bet v 1 antibody and the second anti-Bet v 1 antibody.

42. A method of treating one or more symptoms of birch allergy in a subject, the method comprising subcutaneously administering to the subject a pharmaceutical composition that consists essentially of a first anti-Bet v 1 antibody at a dose of about 300 mg and a second anti-Bet v 1 antibody at a dose of about 300 mg, wherein the first anti-Bet v 1 antibody is bremzalerbart and the second anti-Bet v 1 antibody is atisnolerbart.

43. A method of treating one or more signs or symptoms associated with allergic conjunctivitis in a subject having birch tree pollen allergy, the method comprising subcutaneously administering to the subject a pharmaceutical composition that consists essentially of a first anti-Bet v 1 antibody at a dose of about 300 mg and a second anti-Bet v 1 antibody at a dose of about 300 mg, wherein the first anti-Bet v 1 antibody is bremzalerbart and the second anti-Bet v 1 antibody is atisnolerbart.

44. The method of claim 42, wherein the subject is an adult.

45. The method of claim 42, wherein the subject:
(i) has a baseline serum allergen-specific IgE level ≥0.7 kUa/L for birch tree pollen and/or Bet v 1 allergen;
(ii) has a baseline birch skin prick test (SPT) mean wheal diameter ≥5 mm;
(iii) has a baseline ocular itch score ≥2 in both eyes after a conjunctival allergen challenge (CAC), as measured using the Ora Calibra® Conjunctival Allergen Challenge Ocular Itching Scale; and/or
(iv) has a baseline conjunctival redness score ≥2 in both eyes after a CAC, as measured using the Ora Calibra® Ocular Hyperemia Scale.

46. The method of claim 43, wherein the subject is an adult.

47. The method of claim 43, wherein the subject:
(i) has a baseline serum allergen-specific IgE level ≥0.7 kUa/L for birch tree pollen and/or Bet v 1 allergen;
(ii) has a baseline birch skin prick test (SPT) mean wheal diameter ≥5 mm;
(iii) has a baseline ocular itch score ≥2 in both eyes after a conjunctival allergen challenge (CAC), as measured using the Ora Calibra® Conjunctival Allergen Challenge Ocular Itching Scale; and/or
(iv) has a baseline conjunctival redness score ≥2 in both eyes after a CAC, as measured using the Ora Calibra® Ocular Hyperemia Scale.


SEQUENCE LISTING (32 sequences)

SEQ ID NO: 1 - protein, 116 aa, synthetic construct
  REGION 1..116: note = Synthetic
QVQLQESGPGLVKPSETLSLTCSVSGGSITNYFWTWIRQSPGKGLEWIGYIYYSGGTNYNPSLKSRVTISIDTSKNQFSLNMNSVTAADTAVYYCAGSYYYGVDVWGQGTTVTVSS

SEQ ID NO: 2 - protein, 8 aa, synthetic construct
  REGION 1..8: note = Synthetic
GGSITNYF

SEQ ID NO: 3 - protein, 7 aa, synthetic construct
  REGION 1..7: note = Synthetic
IYYSGGT

SEQ ID NO: 4 - protein, 10 aa, synthetic construct
  REGION 1..10: note = Synthetic
AGSYYYGVDV

SEQ ID NO: 5 - protein, 107 aa, synthetic construct
  REGION 1..107: note = Synthetic
EIVLTQSPATLSLSPGERATLSCRASQSIKSFLAWYRQKPGQAPRLLIYDASNRPTGIPARFSGSGSGTDFTLTINSLESEDFAVYFCQQRNNWPFTFGPGTKVDIK

SEQ ID NO: 6 - protein, 6 aa, synthetic construct
  REGION 1..6: note = Synthetic
QSIKSF

SEQ ID NO: 7 - skipped

SEQ ID NO: 8 - protein, 9 aa, synthetic construct
  REGION 1..9: note = Synthetic
QQRNNWPFT

SEQ ID NO: 9 - protein, 443 aa, synthetic construct
  REGION 1..443: note = Synthetic
QVQLQESGPGLVKPSETLSLTCSVSGGSITNYFWTWIRQSPGKGLEWIGYIYYSGGTNYNPSLKSRVTISIDTSKNQFSLNMNSVTAADTAVYYCAGSYYYGVDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
  REGION 1..214: note = Synthetic
EIVLTQSPATLSLSPGERATLSCRASQSIKSFLAWYRQKPGQAPRLLIYDASNRPTGIPARFSGSGSGTDFTLTINSLESEDFAVYFCQQRNNWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 119 aa, synthetic construct
  REGION 1..119: note = Synthetic
EVQLVESGGDLVQPGGSLRLSCAASGFTFSSYEMNWVRQAPGKGLEWVSFISDSSSNIYYADSVKGRFTISRDNAKKSLYLQMTSLRAEDTAVYYCAREAIGSTSFDNWGQGTLVTVSS

SEQ ID NO: 12 - protein, 8 aa, synthetic construct
  REGION 1..8: note = Synthetic
GFTFSSYE

SEQ ID NO: 13 - protein, 8 aa, synthetic construct
  REGION 1..8: note = Synthetic
ISDSSSNI

SEQ ID NO: 14 - protein, 12 aa, synthetic construct
  REGION 1..12: note = Synthetic
AREAIGSTSFDN

SEQ ID NO: 15 - protein, 107 aa, synthetic construct
  REGION 1..107: note = Synthetic
EIVMTQSPATLSVSPGERATLSCRASQSVSSSLAWYQQKPGQAPRRLIYSASTRATGIPARFSGSGSGTEFTLTISSLQSEDFAIYYCHQYNNWPLTFGGGTKVEIK

SEQ ID NO: 16 - protein, 6 aa, synthetic construct
  REGION 1..6: note = Synthetic
QSVSSS

SEQ ID NO: 17 - skipped

SEQ ID NO: 18 - protein, 9 aa, synthetic construct
  REGION 1..9: note = Synthetic
HQYNNWPLT

SEQ ID NO: 19 - protein, 446 aa, synthetic construct
  REGION 1..446: note = Synthetic
EVQLVESGGDLVQPGGSLRLSCAASGFTFSSYEMNWVRQAPGKGLEWVSFISDSSSNIYYADSVKGRFTISRDNAKKSLYLQMTSLRAEDTAVYYCAREAIGSTSFDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 214 aa, synthetic construct
  REGION 1..214: note = Synthetic
EIVMTQSPATLSVSPGERATLSCRASQSVSSSLAWYQQKPGQAPRRLIYSASTRATGIPARFSGSGSGTEFTLTISSLQSEDFAIYYCHQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 120 aa, synthetic construct
  REGION 1..120: note = Synthetic
QVQLVQSGAEVKKPGASVKVSCKASGYTFISYNIFWVRQATGQGLDWMGWMNPFRNNAGYAQKFQGRVTVTWDTSISTAYMELSSLSSEDTAIYYCAREHGSSWGFFDYWGQGTLVTVSS

SEQ ID NO: 22 - protein, 8 aa, synthetic construct
  REGION 1..8: note = Synthetic
GYTFISYN

SEQ ID NO: 23 - protein, 8 aa, synthetic construct
  REGION 1..8: note = Synthetic
MNPFRNNA

SEQ ID NO: 24 - protein, 13 aa, synthetic construct
  REGION 1..13: note = Synthetic
AREHGSSWGFFDY

SEQ ID NO: 25 - protein, 108 aa, synthetic construct
  REGION 1..108: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 26 - protein, 7 aa, synthetic construct
  REGION 1..7: note = Synthetic
QSVSSSY

SEQ ID NO: 27 - skipped

SEQ ID NO: 28 - protein, 9 aa, synthetic construct
  REGION 1..9: note = Synthetic
QQYGSSPWT

SEQ ID NO: 29 - protein, 447 aa, synthetic construct
  REGION 1..447: note = Synthetic
QVQLVQSGAEVKKPGASVKVSCKASGYTFISYNIFWVRQATGQGLDWMGWMNPFRNNAGYAQKFQGRVTVTWDTSISTAYMELSSLSSEDTAIYYCAREHGSSWGFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 215 aa, synthetic construct
  REGION 1..215: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 160 aa, synthetic construct
  REGION 1..160: note = Synthetic
MGVFNYETETTSVIPAARLFKAFILDGDNLFPKVAPQAISSVENIEGNGGPGTIKKISFPEGLPFKYVKDRVDEVDHTNFKYNYSVIEGGPIGDTLEKISNEIKIVATPDGGSILKISNKYHTKGDHEVKAEQVKASKEMGETLLRAVESYLLAHSDAYN

SEQ ID NO: 32 - protein, 160 aa, synthetic construct
  REGION 1..160: note = Synthetic
MGVFNYETETTSVIPAARLFKAFILDGDNLFPKVAPQAISSVENIEGNGGPGTIKKISFPEGFPFKYVKDRVDEVDHTNFKYNYSVIEGGPIGDTLEKISNEIKIVATPDGGSILKISNKYHTKGDHEVKAEQVKASKEMGETLLRAVESYLLAHSDAYN